(19) United States
(12) Patent Application Publication
(10) Pub. No.: US 2023/0201061 A1
(43) Pub. Date: Jun. 29, 2023

(54) POWER ASSISTED MOBILITY FOR SURGICAL TABLE

(71) Applicant: Auris Health, Inc., Redwood City, CA (US)

(72) Inventors: Bryan James CULVER, Mountain View, CA (US); Travis C. COVINGTON, Campbell, CA (US); Aren Calder HILL, Mountain View, CA (US); Sven WEHRMANN, Redwood City, CA (US)

(21) Appl. No.: 18/116,255

(22) Filed: Mar. 1, 2023

Related U.S. Application Data
(63) Continuation of application No. PCT/IB2021/058606, filed on Sep. 21, 2021.
(60) Provisional application No. 63/086,043, filed on Sep. 30, 2020.

(51) Int. Cl. A61G 13/10

(52) U.S. Cl. CPC A61G 13/104

(57) ABSTRACT

Certain aspects relate to systems and techniques for a mobile medical platform that includes a rigid base and one or more wheel assemblies coupled to a first side of the rigid base to support and move the rigid base in a physical environment. A respective wheel assembly of the one or more wheel assemblies includes a wheel, a first motor configured to steer the wheel, and a second motor configured to roll the wheel. The wheel is configured to respectively rotate around a first axis and a second axis that is different from the first axis. The first motor is positioned around a respective one of the first axis and the second axis, and the first axis is aligned with the second axis that results in a negligible caster angle of the wheel. A method of operating the mobile medical platform is also disclosed.

### RELATED APPLICATIONS

This application is a continuation of PCT International Patent Application No. PCT/IB2021/058606, filed on Sep. 21, 2021, entitled “Power Assisted Mobility for Surgical Table,” which claims priority to U.S. Provisional Patent Application No. 63/086,043, filed on Sep. 30, 2020, entitled “Power Assisted Mobility for Surgical Table,” all of which are incorporated by reference herein in their entireties.

### TECHNICAL FIELD

The systems and methods disclosed herein are directed to power assisted mechanisms, and more particularly to power assisted mechanisms for transporting medical platforms.

### BACKGROUND

Medical platforms, such as surgical tables or hospital beds, can be used to support a patient during a medical procedure. Such medical platforms need to be moved from time to time within a hospital to facilitate optimal hospital operations and logistics. Conventionally, such medical platforms are moved manually.

However, medical platforms that are loaded with other devices are heavier than conventional beds, and transporting such medical platforms can be challenging.

### SUMMARY

There is a need for power-assisted mechanism for transporting medical platforms. Disclosed herein is a power-assisted mobile medical platform for a surgical or medical robotics system. Such medical platform facilitates transportation into different medical settings, fulfilling many needs, such as serving as a surgical table or a hospital bed. In addition, a medical platform with power assisted mobility allows for precise movement of the medical platform during transport or for placement. For example, a medical platform with power-assisted mobility may be able to maneuver around a tight corner that a conventional hospital bed requiring manual maneuvering may not be able to easily navigate. In another example, a medical platform with power-assisted mobility may be precisely positioned in an exact location, such as in an optimal position in a surgical suite.

In accordance with some embodiments, a mobile medical platform includes a rigid base and one or more wheel assemblies that are coupled to a first side of the rigid base to support and move the rigid base in a physical environment. A respective wheel assembly of the one or more wheel assemblies includes a wheel, a first motor configured to steer the wheel, and a second motor configured to roll the wheel. The combination of the two motors per wheel provides two degrees of freedom (e.g., for propulsion and steering) per wheel, which facilitates accurate positioning of the mobile medical platform and also enables power-assisted maneuvers that are not possible with conventional beds.

In some embodiments, the first motor is configured to steer the wheel around a first axis that is substantially perpendicular to a plane corresponding to the first side of the rigid base and the second motor is configured to roll the wheel around a second axis that is substantially parallel to the plane corresponding to the first side of the rigid base.

In some embodiments, the first axis and the second axis are aligned to substantially eliminate a caster angle of the first axis.

In some embodiments, the wheel assembly also includes a first spring that is positioned to exert a downward force on the wheel.

In some embodiments, the wheel assembly also includes a second spring that is positioned to dampen relative movement between the wheel and the rigid base.

In some embodiments, the wheel assembly includes a first spring and a second spring that is located below the first spring and above the wheel. The first spring has a greater spring constant than the second spring.

In some embodiments, the mobile medical platform includes one or more processors and memory storing instructions which, when executed by the one or more processors, cause at least one of the first motor or the second motor to move the wheel in accordance with one or more inputs.

In some embodiments, the mobile medical platform includes at least two wheel assemblies and the stored instructions, when executed by the one or more processors, cause the one or more processors to, in accordance with a determination that preset automatic-braking criteria are met, trigger respective first motors of one or more of the at least two wheel assemblies to steer the respective wheels of the at least two wheel assemblies into a preset braking configuration.

In some embodiments, the mobile medical platform includes at least four wheel assemblies, and the preset braking configuration includes rolling axes of adjacent wheels of the four wheel assemblies being arranged at different angles.

In some embodiments, the stored instructions, when executed by the one or more processors, cause the one or more processors to trigger the at least two wheel assemblies to align respective wheels of the at least two wheel assemblies in a common direction in accordance with a determination that first criteria are met, and trigger the at least two wheel assemblies to place the respective wheels of the at least two wheel assemblies in a preset braking configuration in accordance with a determination that the first criteria are not met.

In some embodiments, the first criteria includes a requirement that an input of a first preset type is continuously maintained in order for the first criteria to be met. For example, the first criteria may require that a dead man switch is pressed at during movement and operation of the mobile medical platform.

In some embodiments, the stored instructions, when executed by the one or more processors, cause the one or more processors to receive one or more control parameters corresponding to user from one or more input devices that are in communication with the one or more processors, and control respective first motors and respective second motors of the one or more wheel assemblies to move respective wheels in accordance the one or more control parameters.

In some embodiments, controlling the respective first motors and the respective second motors of the one or more wheel assemblies to move the respective wheels in accordance the one or more control parameters includes controlling the first motor and the second motor of the respective wheel assembly to steer and roll the wheel of the respective wheel assembly at a same time.

In some embodiments, the stored instructions, when executed by the one or more processors, cause the one or more processors to coordinate operations of two or more wheel assemblies to achieve a requested movement of the rigid base.

In some embodiments, the mobile medical platform includes a robotic surgery system that is coupled to the rigid base. The robotic surgery system includes a table top that is supported by the rigid base and one or more robotic arms that are configured to move relative to the table top.

In accordance with some embodiments, a mobile medical platform includes a rigid base and one or more wheel assemblies that are coupled to a first side of the rigid base and support the rigid base. A respective wheel assembly of the one or more wheel assemblies includes a wheel that is configured to respectively rotate around a first axis and a second axis that is different from the first axis. The first motor is positioned for rotating the wheel around a respective one of the first axis and the second axis. The first axis is aligned with the second axis, resulting in a negligible caster angle of the wheel. The alignment of the first axis and the second axis facilitates accurate positioning of the mobile medical platform by reducing or eliminating the swept volume associated with the caster. It also facilitates independent selection of the steering direction for each wheel, which simplifies the control mechanism and further improves the positioning accuracy.

In some embodiments, the first motor is positioned for rotating the wheel around the first axis and the respective wheel assembly of the one or more wheel assemblies further includes second motor positioned to rotate the wheel around the second axis. The second axis is substantially parallel to a plane corresponding to a first side of the rigid base.

In some embodiments, the respective wheel assembly of the one or more wheel assemblies further includes a first spring positioned to exert a downward force on the wheel.

In some embodiments, the respective wheel assembly of the one or more wheel assemblies further includes a second spring positioned to dampen relative movement between the wheel and the rigid base.

In some embodiments, the respective wheel assembly of the one or more wheel assemblies further includes a first spring and a second spring located below the first spring. The first spring has a greater spring constant than the second spring.

In some embodiments, the mobile medical platform further includes one or more processors and memory storing instructions, which, when executed by the one or more processors, cause the first motor to move the wheel in accordance with one or more inputs.

In some embodiments, the one or more wheel assemblies include at least two wheel assemblies. The stored instructions, when executed by the one or more processors, cause the one or more processors to, in accordance with a determination that preset automatic-braking criteria are met, trigger respective first motors of one or more of the at least two wheel assemblies to rotate respective wheels of the at least two wheel assemblies into a preset braking configuration.

In some embodiments, the one or more wheel assemblies include four wheel assemblies, and the preset braking configuration includes second axes of adjacent wheels of the four wheel assemblies being arranged at different angles.

In some embodiments, the respective wheel assembly further includes a second motor. The stored instructions, when executed by the one or more processors, cause the one or more processors to receive one or more control parameters corresponding to user from one or more input devices that are in communication with the one or more processors, and control respective first motors and respective second motors of the one or more wheel assemblies to move respective wheels of the one or more wheel assemblies in accordance the one or more control parameters.

In some embodiments, the one or more wheel assemblies include at least two wheel assemblies. The stored instructions, when executed by the one or more processors, cause the one or more processors to trigger the at least two wheel assemblies to align respective wheels of the at least two wheel assembles in a common direction in accordance with a determination that first criteria are met, and trigger the at least two wheel assemblies to place the respective wheels of the at least two wheel assembles in a preset braking configuration in accordance with a determination that the first criteria are not met.

In some embodiments, the first criteria include a requirement that an input of a first preset type is continuously maintained in order for the first criteria to be met.

In some embodiments, the respective wheel assembly further includes a second motor. The stored instructions, when executed by the one or more processors, cause the one or more processors to control the first motor and the second motor of the respective wheel assembly to rotate the wheel of the respective wheel assembly around the first axis and the second axis at the same time.

In some embodiments, the one or more wheel assemblies include at least two wheel assemblies. The stored instructions, when executed by the one or more processors, cause the one or more processors to coordinate operations of two or more wheel assemblies to achieve a requested movement of the rigid base.

In some embodiments, the mobile medical platform further includes a robotic surgery system that is coupled to the rigid base. The robotic surgery system includes a table top and one or more robotic arms that are configured to move relative to the table top.

In accordance with some embodiments, a mobile medical platform includes a rigid base and one or more wheel assemblies that are coupled to a first side of the rigid base and support the rigid base during movement of the mobile medical platform. A respective wheel assembly of the one or more wheel assemblies includes a wheel, a first motor positioned for rotating the wheel, a first spring positioned to exert a downward force on the wheel, and a second spring positioned to dampen relative movement between the wheel and the rigid base. The combination of the two springs facilitates that the respective wheel remains in contact with a floor while dampening shocks or vibrations caused by a non-flat floor surface.

In some embodiments, the second spring is located below the first spring, the second spring is located above the wheel, and the first spring has a greater spring constant than the second spring.

In some embodiments, the first motor is positioned for rotating the wheel around a first axis that is substantially perpendicular to a plane corresponding to the first side of the rigid base. The respective wheel assembly of the one or more wheel assemblies further includes a second motor positioned for rolling the wheel around a second axis that is substantially parallel to the plane corresponding to the first side of the rigid base.

In some embodiments, the first axis and the second axis are aligned to substantially eliminate a caster angle of the first axis.

In some embodiments, the mobile medical platform includes one or more processors and memory storing instructions, which, when executed by the one or more processors, cause at least one of the first motor or the second motor to move the wheel in accordance with one or more inputs.

In some embodiments, the one or more wheel assemblies include at least two wheel assemblies. The stored instructions, when executed by the one or more processors, cause the one or more processors to, in accordance with a determination that preset automatic-braking criteria are met, trigger respective first motors of one or more of the at least two wheel assemblies to steer respective wheels of the at least two wheel assemblies into a preset braking configuration.

In some embodiments, the one or more wheel assemblies include four wheel assemblies, and the preset braking configuration includes second axes of adjacent wheels of the four wheel assemblies being arranged at different angles.

In some embodiments, the one or more wheel assemblies include at least two wheel assemblies. The stored instructions, when executed by the one or more processors, cause the one or more processors to trigger the at least two wheel assemblies to align the respective wheels of the at least two wheel assembles in a common direction in accordance with a determination that first criteria are met, and trigger the at least two wheel assemblies to place the respective wheels of the at least two wheel assembles in a preset braking configuration in accordance with a determination that the first criteria are not met.

In some embodiments, the first criteria include a requirement that an input of a first preset type is continuously maintained in order for the first criteria to be met.

In some embodiments, the stored instructions, when executed by the one or more processors, cause the one or more processors to receive one or more control parameters corresponding to user input from one or more input devices that are in communication with the one or more processors, and control the respective first motors and the respective second motors of the one or more wheel assemblies to move respective wheels in accordance the one or more control parameters.

In some embodiments, controlling the respective first motors and the respective second motors of the one or more wheel assemblies to move the respective wheels in accordance the one or more control parameters includes controlling the first motor and the second motor of the respective wheel assembly to steer and roll the wheel of the respective wheel assembly at a same time.

In some embodiments, the stored instructions, when executed by the one or more processors, cause the one or more processors to coordinate operations of two or more wheel assemblies to achieve a requested movement of the rigid base.

In some embodiments, the mobile patient platform includes a robotic surgery system that is coupled to the rigid base. The robotic surgery system includes a table top and one or more robotic arms that are configured to move relative to the table top.

In accordance with some embodiments, a mobile medical platform includes a rigid base and at least four wheel assemblies that are coupled to the rigid base and support the rigid base. A respective wheel assembly of the at least four wheel assemblies includes a respective wheel and a respective first motor positioned for steering the respective wheel. The mobile medical platform also includes one or more processors and memory storing instructions, which, when executed by the one or more processors, cause steering of the respective wheels of the at least four wheel assemblies such that the respective wheels of the at least four wheel assemblies are aligned in a common direction at a first time, and the respective wheels of the at least four wheel assemblies are arranged in a braking configuration, at a second time distinct from the first time, so that the rigid base is immobilized. Such configuration allows rapid and efficient braking of the mobile medical platform, which, in turn, improves the accuracy in positioning the mobile medical platform and the safety in transportation of the mobile medical platform.

In some embodiments, the respective wheels of the at least four wheel assemblies are directed to a common point while in the braking configuration.

In some embodiments, the common point is a centroid of the at least four wheel assemblies.

In some embodiments, the respective first motor is positioned for steering the respective wheel around a first axis that is substantially perpendicular to a plane corresponding to a first side of the rigid base, and the respective wheel assembly of the at least four wheel assemblies further includes a respective second motor positioned for rolling the respective wheel around a second axis that is substantially parallel to the plane corresponding to the first side of the rigid base.

In some embodiments, the first axis and the second axis are aligned to substantially eliminate a caster angle of the first axis.

In some embodiments, the stored instructions, when executed by the one or more processors, cause at least one of the respective first motor or the respective second motor to move the respective wheel in accordance with one or more inputs.

In some embodiments, the stored instructions, when executed by the one or more processors, cause the one or more processors to receive one or more control parameters from one or more input devices that are in communication with the one or more processors, and control the respective first motors and the respective second motors of the one or more wheel assemblies to move the respective wheels in accordance the one or more control parameters.

In some embodiments, controlling the respective first motors and the respective second motors of the one or more wheel assemblies to move the respective wheels in accordance the one or more control parameters includes controlling the respective first motor and the respective second motor of the respective wheel assembly to steer and roll the respective wheel of the respective wheel assembly at a same time.

In some embodiments, the respective wheel assembly of the at least four wheel assemblies further includes a respective first spring that is positioned to exert a downward force on the respective wheel.

In some embodiments, the respective wheel assembly of the at least four wheel assemblies further includes a respective second spring that is positioned to dampen relative movement between the respective wheel and the rigid base.

In some embodiments, the respective wheel assembly of the at least four wheel assemblies further includes a respective first spring and a respective second spring located below the respective first spring. The respective second spring is located above the respective wheel, and the respective first spring has a greater spring constant than a spring constant of the respective second spring.

In some embodiments, the stored instructions, when executed by the one or more processors, cause the one or more processors to, in accordance with a determination that preset automatic-braking criteria are met, trigger respective first motors of one or more of the at least four wheel assemblies to steer the respective wheels of the at least four wheel assemblies into the braking configuration.

In some embodiments, the braking configuration includes second axes of adjacent wheels of the four wheel assemblies being arranged at different angles.

In some embodiments, the stored instructions, when executed by the one or more processors, cause the one or more processors to coordinate operations of two or more wheel assemblies to achieve a requested movement of the rigid base.

In some embodiments, the respective wheels of the at least four wheel assemblies are aligned in a common direction in accordance with a determination that first criteria are met, and the respective wheels of the at least four wheel assemblies are arranged in a braking configuration, in accordance with a determination that the first criteria are not met.

In some embodiments, the first criteria include a requirement that an input of a first preset type is continuously maintained in order for the first criteria to be met.

In some embodiments, a robotic surgery system that is coupled to the rigid base. The robotic surgery system includes a table top and one or more robotic arms that are configured to move relative to the table top.

In accordance with some embodiments, a method performed at a mobile medical platform includes receiving user input to move the mobile medical platform and moving one or more wheel assemblies that are coupled to the rigid base, including activating the first motor to orient the wheel in a direction that corresponds to the user input and activating the second motor to roll the wheel.

In some embodiments, the second motor is activated after the wheel is oriented and the wheel is rolled by the second motor while an orientation of the wheel is maintained in the respective direction.

In some embodiments, the first motor and the second motor are activated at a same time.

In some embodiments, activating the first motor to orient the wheel in a respective direction includes steering, by the first motor, the wheel around a first axis that is substantially perpendicular to a plane corresponding to the first side of the rigid base, and activating the second motor to roll the wheel includes powering the wheel, by the second motor, to roll around a second axis that is substantially parallel to the plane corresponding to the first side of the rigid base.

In some embodiments, the first axis and the second axis are aligned to substantially eliminate a caster angle of the first axis.

In some embodiments, the wheel assembly further includes a first spring that is positioned to exert a downward force on the wheel.

In some embodiments, the wheel assembly further includes a second spring that is positioned to dampen relative movement between the wheel and the rigid base.

In some embodiments, the wheel assembly further includes a first spring and a second spring, the second spring is located above the wheel and below the first spring, and the first spring has a greater spring constant than a spring constant of the second spring.

In some embodiments, the one or more wheel assemblies include at least two wheel assemblies. The method further includes, in accordance with a determination that first criteria are met, triggering at least two wheel assemblies of the one or more wheel assemblies to align the respective wheels of the at least two wheel assembles in a common direction, and in accordance with a determination that first criteria are not met, triggering respective first motors of one or more of the at least two wheel assemblies to steer the respective wheels of the at least two wheel assemblies into a preset braking configuration.

In some embodiments, the one or more wheel assemblies include four wheel assemblies. Triggering respective first motors of one or more of the at least two wheel assemblies to steer the respective wheels of the at least two wheel assemblies into a preset braking configuration includes rotating, by the respective first motors, the respective wheels around the second axes of adjacent wheels of the four wheel assemblies such that second axes of the adjacent wheels of the four wheel assemblies are arranged at different angles.

In some embodiments, the first criteria include a requirement that an input of a first preset type is continuously maintained in order for the first criteria to be met.

In some embodiments, the user input to move the mobile medical platform is received from one or more input devices.

In some embodiments, coordinating operations of two or more wheel assemblies to achieve a requested movement of the rigid base, the requested movement of the rigid base corresponding to the user input.

In some embodiments, the mobile medical platform further includes a robotic surgery system that is coupled to the rigid base. The robotic surgery system includes a table top and one or more robotic arms, and the method further includes moving the one or more robotic arms relative to the table top.

In accordance with some embodiments, a method includes utilizing a mobile medical platform. The mobile medical platform includes at least two wheel assemblies for utilizing the mobile medical platform, wherein the mobile medical platform includes at least two wheel assemblies for receiving input to move the mobile medical platform from one or more input devices, and generating one or more control instructions for controlling respective first motors and respective second motors of the at least two wheel assemblies. Generating the one or more control instructions includes triggering the at least two wheel assemblies to align respective wheels of the at least two wheel assemblies in a common direction in accordance with a determination that the input meets first criteria, and triggering the at least two wheel assemblies to place the respective wheels of the at least two wheel assemblies in a preset braking configuration in accordance with a determination that the input meets second criteria different from the first criteria.

In some embodiments, triggering the at least two wheel assemblies to align the respective wheels of the at least two wheel assemblies in a common direction in accordance with a determination that the input meets first criteria also includes activating the respective first motors to align the respective wheels of the at least two wheel assemblies in a common direction, and activating a respective second motor to roll the respective wheels. The first motor and the second motor are activated at a same time.

In some embodiments, triggering the at least two wheel assemblies to align the respective wheels of the at least two wheel assemblies in a common direction in accordance with a determination that the input meets first criteria includes activating the respective first motors to align the respective wheels of the at least two wheel assemblies in a common direction, and activating the respective second motors to roll the respective wheels after the respective wheels are maintained in the respective direction and while an orientation of the respective wheels are maintained in the respective direction.

In some embodiments, activating the respective first motors to align the respective wheels of the at least two wheel assemblies in a common direction includes steering, by the respective first motors, the respective wheels around respective first axes that are substantially perpendicular to a plane corresponding to the first side of the rigid base. Activating the respective second motors to roll the respective wheels includes powering the wheel, by the respective second motors, to roll around respective second axes that are substantially parallel to the plane corresponding to the first side of the rigid base.

In some embodiments, a respective first axis and respective second axis of a respective wheel of the at least two wheel assemblies are aligned to substantially eliminate a caster angle of the respective first axis.

In some embodiments, the one or more wheel assemblies include at least four wheel assemblies. Triggering the at least four wheel assemblies to place the respective wheels of the at least four wheel assemblies in the preset braking configuration includes rotating, by the respective first motors, the respective wheels around first axes of adjacent wheels of the four wheel assemblies such that the second axes of the adjacent wheels of the four wheel assemblies are arranged at different angles.

In some embodiments, a respective wheel assembly of the two of more wheel assemblies further includes a respective first spring that is positioned to exert a downward force on the respective wheel.

In some embodiments, a respective wheel assembly of the two of more wheel assemblies further includes a respective second spring that is positioned to dampen relative movement between the respective wheel and the rigid base.

In some embodiments, the a respective wheel assembly of the two of more wheel assemblies further includes a respective first spring and a respective second spring. The respective second spring is located above the respective wheel and below the respective first spring, and the respective first spring has a greater spring constant than a spring constant of the respective second spring.

In some embodiments, the method further includes coordinating operations of the at least two wheel assemblies to achieve a requested movement of the rigid base. The requested movement of the rigid base corresponds to the input.

In some embodiments, the mobile medical platform further includes a robotic surgery system that is coupled to the rigid base. The robotic surgery system includes a table top and one or more robotic arms, and the method further includes moving the one or more robotic arms relative to the table top.

### BRIEF DESCRIPTION OF THE DRAWINGS

The disclosed aspects will hereinafter be described in conjunction with the appended drawings, provided to illustrate and not to limit the disclosed aspects, wherein like designations denote like elements.

FIG. 1 illustrates an embodiment of a cart-based robotic system arranged for diagnostic and/or therapeutic bronchoscopy procedure(s).

FIG. 2 depicts further aspects of the robotic system of FIG. 1.

FIG. 3 illustrates an embodiment of the robotic system of FIG. 1 arranged for ureteroscopy.

FIG. 4 illustrates an embodiment of the robotic system of FIG. 1 arranged for a vascular procedure.

FIG. 5 illustrates an embodiment of a table-based robotic system arranged for a bronchoscopy procedure.

FIG. 6 provides an alternative view of the robotic system of FIG. 5.

FIG. 7 illustrates an example system configured to stow robotic arm(s).

FIG. 8 illustrates an embodiment of a table-based robotic system configured for a ureteroscopy procedure.

FIG. 9 illustrates an embodiment of a table-based robotic system configured for a laparoscopic procedure.

FIG. 10 illustrates an embodiment of the table-based robotic system of FIGS. 5-9 with pitch or tilt adjustment.

FIG. 11 provides a detailed illustration of the interface between the table and the column of the table-based robotic system of FIGS. 5-10.

FIG. 12 illustrates an alternative embodiment of a table-based robotic system.

FIG. 13 illustrates an end view of the table-based robotic system of FIG. 12.

FIG. 14 illustrates an end view of a table-based robotic system with robotic arms attached thereto.

FIG. 15 illustrates an exemplary instrument driver.

FIG. 16 illustrates an exemplary medical instrument with a paired instrument driver.

FIG. 17 illustrates an alternative design for an instrument driver and instrument where the axes of the drive units are parallel to the axis of the elongated shaft of the instrument.

FIG. 18 illustrates an instrument having an instrument-based insertion architecture.

FIG. 19 illustrates an exemplary controller.

FIG. 20 depicts a block diagram illustrating a localization system that estimates a location of one or more elements of the robotic systems of FIGS. 1-10, such as the location of the instrument of FIGS. 16-18, in accordance to an example embodiment.

FIG. 21 illustrates an embodiment of a mobile medical platform that includes one or more wheel assemblies in accordance with some embodiments.

FIG. 22A illustrates a bottom view of the mobile medical platform of FIG. 21 in accordance with some embodiments.

FIG. 22B illustrates a bottom view of a mobile medical platform in accordance with some embodiments.

FIG. 23A illustrates a perspective view of a wheel assembly of the mobile medical platform of FIG. 21 in accordance with some embodiments.

FIG. 23B illustrates a cross-sectional view of the wheel assembly of FIG. 23A in accordance with some embodiments.

FIG. 23C illustrates a side view of the wheel assembly of FIGS. 23A and 23B in accordance with some embodiments.

FIG. 24A illustrates examples of turning paths of the mobile medical platform of FIG. 21 in accordance with some embodiments.

FIGS. 24B-24D illustrate an example of pivoting the mobile medical platform of FIG. 21 in accordance with some embodiments.

FIG. 24E illustrates an example of precise movement of the mobile medical platform of FIG. 21 in accordance with some embodiments.

FIG. 25A illustrates an embodiment of an input device for controlling movement of the mobile medical platform of FIG. 21 in accordance with some embodiments.

FIG. 25B illustrates a preset braking configuration of the mobile medical platform of FIG. 22B in accordance with some embodiments.

FIG. 25C illustrates a preset braking configuration of the mobile medical platform of FIG. 22A in accordance with some embodiments.

FIGS. 26A-26D show a flowchart illustrating a method performed by a mobile medical platform in accordance with some embodiments.

FIG. 27 is a flowchart illustrating another method performed by a mobile medical platform in accordance with some embodiments.

FIG. 28 is a schematic diagram illustrating electronic components of a mobile medical platform in accordance with some embodiments.

### DETAILED DESCRIPTION

### 1. Overview.

Aspects of the present disclosure may be integrated into a robotically-enabled medical system capable of performing a variety of medical procedures, including both minimally invasive, such as laparoscopy, and non-invasive, such as endoscopy, procedures. Among endoscopy procedures, the system may be capable of performing bronchoscopy, ureteroscopy, gastroscopy, etc.

In addition to performing the breadth of procedures, the system may provide additional benefits, such as enhanced imaging and guidance to assist the physician. Additionally, the system may provide the physician with the ability to perform the procedure from an ergonomic position without the need for awkward arm motions and positions. Still further, the system may provide the physician with the ability to perform the procedure with improved ease of use such that one or more of the instruments of the system can be controlled by a single user.

Various embodiments will be described below in conjunction with the drawings for purposes of illustration. It should be appreciated that many other implementations of the disclosed concepts are possible, and various advantages can be achieved with the disclosed implementations. Headings are included herein for reference and to aid in locating various sections. These headings are not intended to limit the scope of the concepts described with respect thereto. Such concepts may have applicability throughout the entire specification.

### A. Robotic System—Cart.

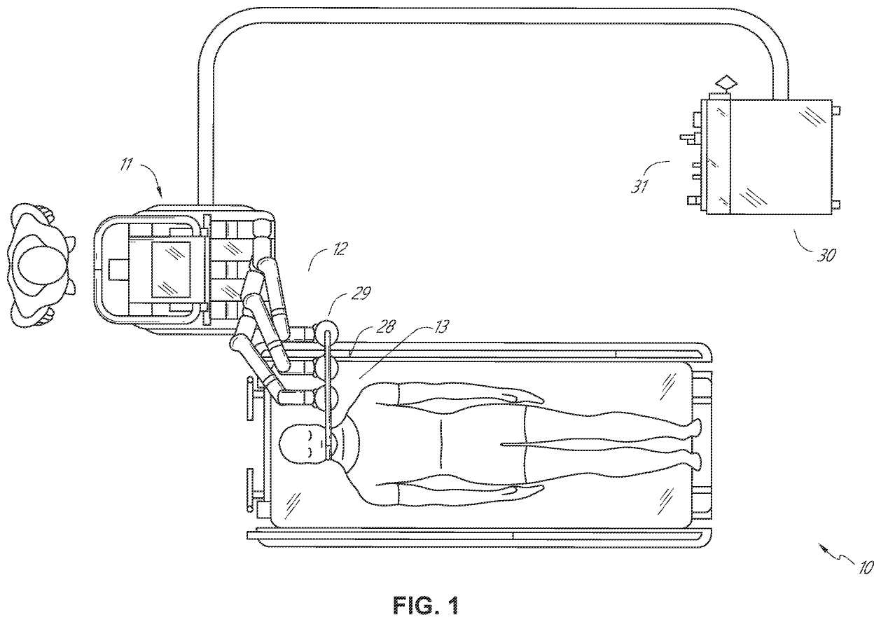

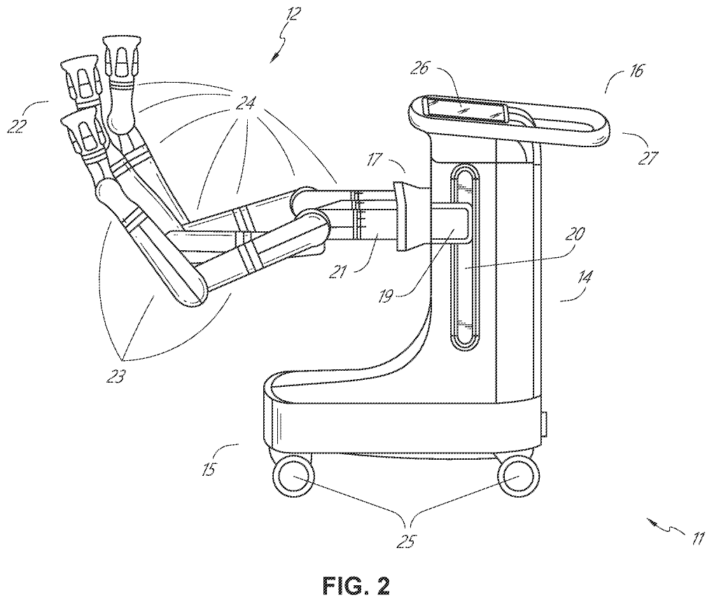

The robotically-enabled medical system may be configured in a variety of ways depending on the particular procedure. FIG. 1 illustrates an embodiment of a cart-based robotically-enabled system 10 arranged for a diagnostic and/or therapeutic bronchoscopy procedure. During a bronchoscopy, the system 10 may comprise a cart 11 having one or more robotic arms 12 to deliver a medical instrument, such as a steerable endoscope 13, which may be a procedure-specific bronchoscope for bronchoscopy, to a natural orifice access point (i.e., the mouth of the patient positioned on a table in the present example) to deliver diagnostic and/or therapeutic tools. As shown, the cart 11 may be positioned proximate to the patient's upper torso in order to provide access to the access point. Similarly, the robotic arms 12 may be actuated to position the bronchoscope relative to the access point. The arrangement in FIG. 1 may also be utilized when performing a gastro-intestinal (GI) procedure with a gastroscope, a specialized endoscope for GI procedures. FIG. 2 depicts an example embodiment of the cart in greater detail.

With continued reference to FIG. 1, once the cart 11 is properly positioned, the robotic arms 12 may insert the steerable endoscope 13 into the patient robotically, manually, or a combination thereof. As shown, the steerable endoscope 13 may comprise at least two telescoping parts, such as an inner leader portion and an outer sheath portion, each portion coupled to a separate instrument driver from the set of instrument drivers 28, each instrument driver coupled to the distal end of an individual robotic arm. This linear arrangement of the instrument drivers 28, which facilitates coaxially aligning the leader portion with the sheath portion, creates a “virtual rail” 29 that may be repositioned in space by manipulating the one or more robotic arms 12 into different angles and/or positions. The virtual rails described herein are depicted in the Figures using dashed lines, and accordingly the dashed lines do not depict any physical structure of the system. Translation of the instrument drivers 28 along the virtual rail 29 telescopes the inner leader portion relative to the outer sheath portion or advances or retracts the endoscope 13 from the patient. The angle of the virtual rail 29 may be adjusted, translated, and pivoted based on clinical application or physician preference. For example, in bronchoscopy, the angle and position of the virtual rail 29 as shown represents a compromise between providing physician access to the endoscope 13 while minimizing friction that results from bending the endoscope 13 into the patient's mouth.

The endoscope 13 may be directed down the patient's trachea and lungs after insertion using precise commands from the robotic system until reaching the target destination or operative site. In order to enhance navigation through the patient's lung network and/or reach the desired target, the endoscope 13 may be manipulated to telescopically extend the inner leader portion from the outer sheath portion to obtain enhanced articulation and greater bend radius. The use of separate instrument drivers 28 also allows the leader portion and sheath portion to be driven independent of each other.

For example, the endoscope 13 may be directed to deliver a biopsy needle to a target, such as, for example, a lesion or nodule within the lungs of a patient. The needle may be deployed down a working channel that runs the length of the endoscope to obtain a tissue sample to be analyzed by a pathologist. Depending on the pathology results, additional tools may be deployed down the working channel of the endoscope for additional biopsies. After identifying a nodule to be malignant, the endoscope 13 may endoscopically deliver tools to resect the potentially cancerous tissue. In some instances, diagnostic and therapeutic treatments can be delivered in separate procedures. In those circumstances, the endoscope 13 may also be used to deliver a fiducial to “mark” the location of the target nodule as well. In other instances, diagnostic and therapeutic treatments may be delivered during the same procedure.

The system 10 may also include a movable tower 30, which may be connected via support cables to the cart 11 to provide support for controls, electronics, fluidics, optics, sensors, and/or power to the cart 11. Placing such functionality in the tower 30 allows for a smaller form factor cart 11 that may be more easily adjusted and/or re-positioned by an operating physician and his/her staff. Additionally, the division of functionality between the cart/table and the support tower 30 reduces operating room clutter and facilitates improving clinical workflow. While the cart 11 may be positioned close to the patient, the tower 30 may be stowed in a remote location to stay out of the way during a procedure.

In support of the robotic systems described above, the tower 30 may include component(s) of a computer-based control system that stores computer program instructions, for example, within a non-transitory computer-readable storage medium such as a persistent magnetic storage drive, solid state drive, etc. The execution of those instructions, whether the execution occurs in the tower 30 or the cart 11, may control the entire system or sub-system(s) thereof. For example, when executed by a processor of the computer system, the instructions may cause the components of the robotics system to actuate the relevant carriages and arm mounts, actuate the robotics arms, and control the medical instruments. For example, in response to receiving the control signal, the motors in the joints of the robotics arms may position the arms into a certain posture.

The tower 30 may also include a pump, flow meter, valve control, and/or fluid access in order to provide controlled irrigation and aspiration capabilities to the system that may be deployed through the endoscope 13. These components may also be controlled using the computer system of tower 30. In some embodiments, irrigation and aspiration capabilities may be delivered directly to the endoscope 13 through separate cable(s).

The tower 30 may include a voltage and surge protector designed to provide filtered and protected electrical power to the cart 11, thereby avoiding placement of a power transformer and other auxiliary power components in the cart 11, resulting in a smaller, more moveable cart 11.

The tower 30 may also include support equipment for the sensors deployed throughout the robotic system 10. For example, the tower 30 may include opto-electronics equipment for detecting, receiving, and processing data received from the optical sensors or cameras throughout the robotic system 10. In combination with the control system, such opto-electronics equipment may be used to generate real-time images for display in any number of consoles deployed throughout the system, including in the tower 30. Similarly, the tower 30 may also include an electronic subsystem for receiving and processing signals received from deployed electromagnetic (EM) sensors. The tower 30 may also be used to house and position an EM field generator for detection by EM sensors in or on the medical instrument.

The tower 30 may also include a console 31 in addition to other consoles available in the rest of the system, e.g., console mounted on top of the cart. The console 31 may include a user interface and a display screen, such as a touchscreen, for the physician operator. Consoles in system 10 are generally designed to provide both robotic controls as well as pre-operative and real-time information of the procedure, such as navigational and localization information of the endoscope 13. When the console 31 is not the only console available to the physician, it may be used by a second operator, such as a nurse, to monitor the health or vitals of the patient and the operation of system, as well as provide procedure-specific data, such as navigational and localization information. In other embodiments, the console 31 is housed in a body that is separate from the tower 30.

The tower 30 may be coupled to the cart 11 and endoscope 13 through one or more cables or connections (not shown). In some embodiments, the support functionality from the tower 30 may be provided through a single cable to the cart 11, simplifying and de-cluttering the operating room. In other embodiments, specific functionality may be coupled in separate cabling and connections. For example, while power may be provided through a single power cable to the cart, the support for controls, optics, fluidics, and/or navigation may be provided through a separate cable.

FIG. 2 provides a detailed illustration of an embodiment of the cart from the cart-based robotically-enabled system shown in FIG. 1. The cart 11 generally includes an elongated support structure 14 (often referred to as a “column”), a cart base 15, and a console 16 at the top of the column 14. The column 14 may include one or more carriages, such as a carriage 17 (alternatively “arm support”) for supporting the deployment of one or more robotic arms 12 (three shown in FIG. 2). The carriage 17 may include individually configurable arm mounts that rotate along a perpendicular axis to adjust the base of the robotic arms 12 for better positioning relative to the patient. The carriage 17 also includes a carriage interface 19 that allows the carriage 17 to vertically translate along the column 14.

The carriage interface 19 is connected to the column 14 through slots, such as slot 20, that are positioned on opposite sides of the column 14 to guide the vertical translation of the carriage 17. The slot 20 contains a vertical translation interface to position and hold the carriage at various vertical heights relative to the cart base 15. Vertical translation of the carriage 17 allows the cart 11 to adjust the reach of the robotic arms 12 to meet a variety of table heights, patient sizes, and physician preferences. Similarly, the individually configurable arm mounts on the carriage 17 allow the robotic arm base 21 of robotic arms 12 to be angled in a variety of configurations.

In some embodiments, the slot 20 may be supplemented with slot covers that are flush and parallel to the slot surface to prevent dirt and fluid ingress into the internal chambers of the column 14 and the vertical translation interface as the carriage 17 vertically translates. The slot covers may be deployed through pairs of spring spools positioned near the vertical top and bottom of the slot 20. The covers are coiled within the spools until deployed to extend and retract from their coiled state as the carriage 17 vertically translates up and down. The spring-loading of the spools provides force to retract the cover into a spool when carriage 17 translates towards the spool, while also maintaining a tight seal when the carriage 17 translates away from the spool. The covers may be connected to the carriage 17 using, for example, brackets in the carriage interface 19 to ensure proper extension and retraction of the cover as the carriage 17 translates.

The column 14 may internally comprise mechanisms, such as gears and motors, that are designed to use a vertically aligned lead screw to translate the carriage 17 in a mechanized fashion in response to control signals generated in response to user inputs, e.g., inputs from the console 16.

The robotic arms 12 may generally comprise robotic arm bases 21 and end effectors 22, separated by a series of linkages 23 that are connected by a series of joints 24, each joint comprising an independent actuator, each actuator comprising an independently controllable motor. Each independently controllable joint represents an independent degree of freedom available to the robotic arm. Each of the arms 12 have seven joints, and thus provide seven degrees of freedom. A multitude of joints result in a multitude of degrees of freedom, allowing for “redundant” degrees of freedom. Redundant degrees of freedom allow the robotic arms 12 to position their respective end effectors 22 at a specific position, orientation, and trajectory in space using different linkage positions and joint angles. This allows for the system to position and direct a medical instrument from a desired point in space while allowing the physician to move the arm joints into a clinically advantageous position away from the patient to create greater access, while avoiding arm collisions.

The cart base 15 balances the weight of the column 14, carriage 17, and arms 12 over the floor. Accordingly, the cart base 15 houses heavier components, such as electronics, motors, power supply, as well as components that either enable movement and/or immobilize the cart. For example, the cart base 15 includes one or more wheel assemblies that allow for the cart to easily move around the room prior to a procedure. After reaching the appropriate position, wheels of the one of more wheel assemblies may be immobilized using wheel locks or may be arranged in a preset braking configuration that maintains the cart 11 immobilized during the procedure.

Positioned at the vertical end of column 14, the console 16 allows for both a user interface for receiving user input and a display screen (or a dual-purpose device such as, for example, a touchscreen 26) to provide the physician user with both pre-operative and intra-operative data. Potential pre-operative data on the touchscreen 26 may include pre-operative plans, navigation and mapping data derived from pre-operative computerized tomography (CT) scans, and/or notes from pre-operative patient interviews. Intra-operative data on display may include optical information provided from the tool, sensor and coordinate information from sensors, as well as vital patient statistics, such as respiration, heart rate, and/or pulse. The console 16 may be positioned and tilted to allow a physician to access the console from the side of the column 14 opposite carriage 17. From this position, the physician may view the console 16, robotic arms 12, and patient while operating the console 16 from behind the cart 11. As shown, the console 16 also includes a handle 27 to assist with maneuvering and stabilizing cart 11.

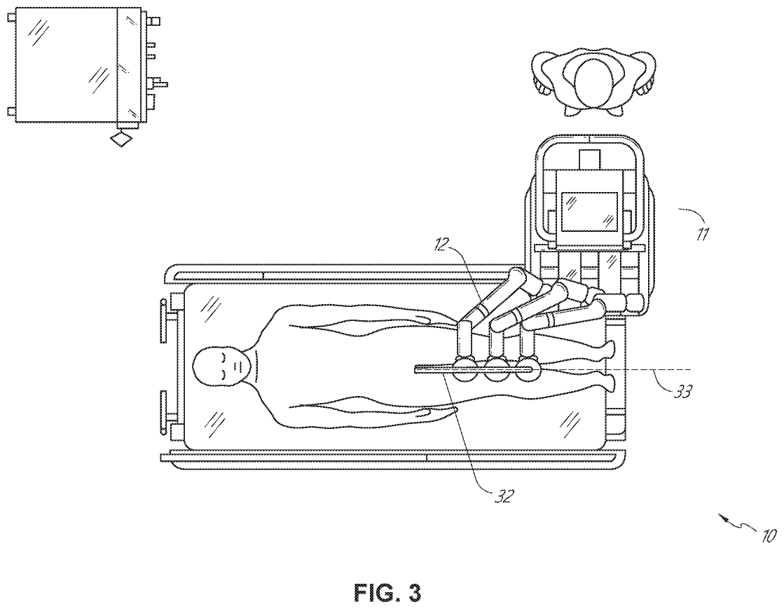

FIG. 3 illustrates an embodiment of a robotically-enabled system 10 arranged for ureteroscopy. In a ureteroscopic procedure, the cart 11 may be positioned to deliver a ureteroscope 32, a procedure-specific endoscope designed to traverse a patient's urethra and ureter, to the lower abdominal area of the patient. In a ureteroscopy, it may be desirable for the ureteroscope 32 to be directly aligned with the patient's urethra to reduce friction and forces on the sensitive anatomy in the area. As shown, the cart 11 may be aligned at the foot of the table to allow the robotic arms 12 to position the ureteroscope 32 for direct linear access to the patient's urethra. From the foot of the table, the robotic arms 12 may insert the ureteroscope 32 along the virtual rail 33 directly into the patient's lower abdomen through the urethra.

After insertion into the urethra, using similar control techniques as in bronchoscopy, the ureteroscope 32 may be navigated into the bladder, ureters, and/or kidneys for diagnostic and/or therapeutic applications. For example, the ureteroscope 32 may be directed into the ureter and kidneys to break up kidney stone build up using a laser or ultrasonic lithotripsy device deployed down the working channel of the ureteroscope 32. After lithotripsy is complete, the resulting stone fragments may be removed using baskets deployed down the ureteroscope 32.

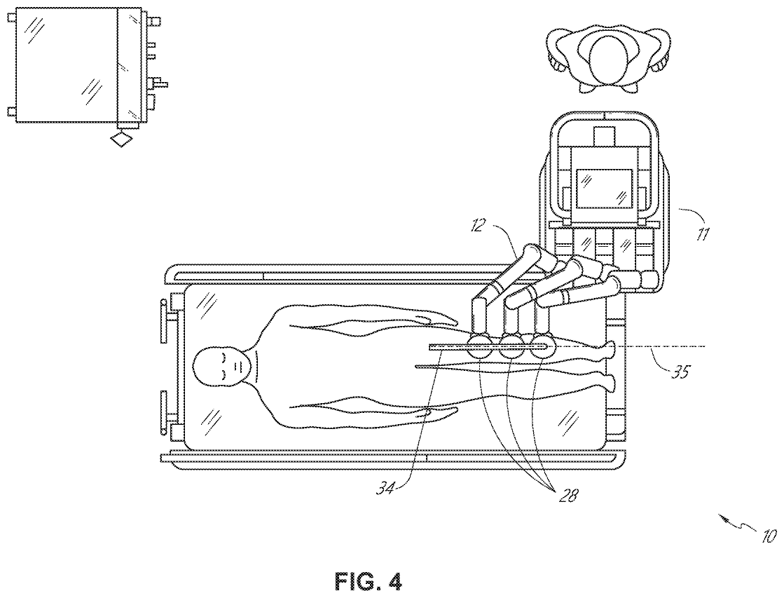

FIG. 4 illustrates an embodiment of a robotically-enabled system similarly arranged for a vascular procedure. In a vascular procedure, the system 10 may be configured such that the cart 11 may deliver a medical instrument 34, such as a steerable catheter, to an access point in the femoral artery in the patient's leg. The femoral artery presents both a larger diameter for navigation as well as a relatively less circuitous and tortuous path to the patient's heart, which simplifies navigation. As in a ureteroscopic procedure, the cart 11 may be positioned towards the patient's legs and lower abdomen to allow the robotic arms 12 to provide a virtual rail 35 with direct linear access to the femoral artery access point in the patient's thigh/hip region. After insertion into the artery, the medical instrument 34 may be directed and inserted by translating the instrument drivers 28. Alternatively, the cart may be positioned around the patient's upper abdomen in order to reach alternative vascular access points, such as, for example, the carotid and brachial arteries near the shoulder and wrist.

### B. Robotic System—Table.

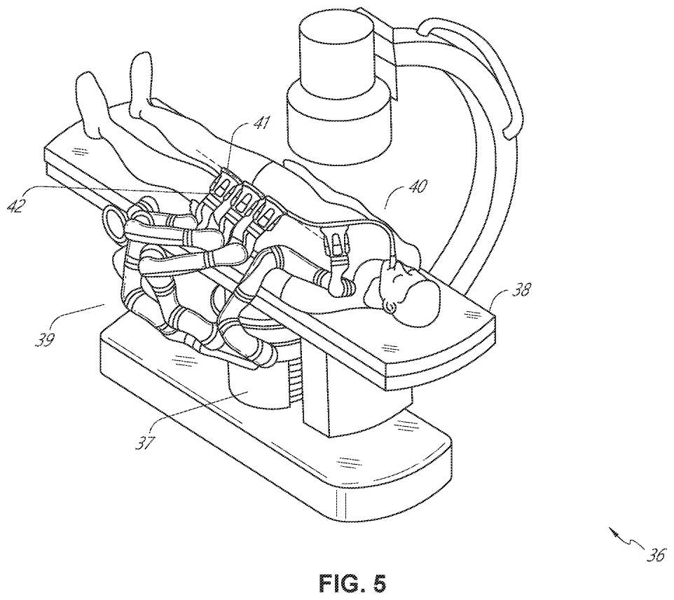

Embodiments of the robotically-enabled medical system may also incorporate the patient's table. Incorporation of the table reduces the amount of capital equipment within the operating room by removing the cart, which allows greater access to the patient. FIG. 5 illustrates an embodiment of such a robotically-enabled system arranged for a bronchoscopy procedure. System 36 includes a support structure or column 37 for supporting platform 38 (shown as a “table” or “bed”) over the floor. Much like in the cart-based systems, the end effectors of the robotic arms 39 of the system 36 comprise instrument drivers 42 that are designed to manipulate an elongated medical instrument, such as a bronchoscope 40 in FIG. 5, through or along a virtual rail 41 formed from the linear alignment of the instrument drivers 42. In practice, a C-arm for providing fluoroscopic imaging may be positioned over the patient's upper abdominal area by placing the emitter and detector around table 38.

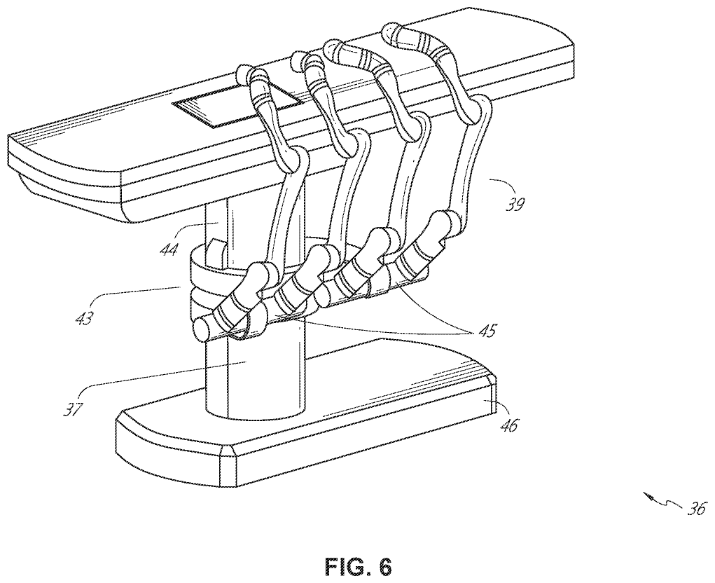

FIG. 6 provides an alternative view of the system 36 without the patient and medical instrument for discussion purposes. As shown, the column 37 may include one or more carriages 43 shown as ring-shaped in the system 36, from which the one or more robotic arms 39 may be based. The carriages 43 may translate along a vertical column interface 44 that runs the length of the column 37 to provide different vantage points from which the robotic arms 39 may be positioned to reach the patient. The carriage(s) 43 may rotate around the column 37 using a mechanical motor positioned within the column 37 to allow the robotic arms 39 to have access to multiples sides of the table 38, such as, for example, both sides of the patient. In embodiments with multiple carriages, the carriages may be individually positioned on the column and may translate and/or rotate independent of the other carriages. While carriages 43 need not surround the column 37 or even be circular, the ring-shape as shown facilitates rotation of the carriages 43 around the column 37 while maintaining structural balance. Rotation and translation of the carriages 43 allows the system to align the medical instruments, such as endoscopes and laparoscopes, into different access points on the patient. In other embodiments (not shown), the system 36 can include a patient table or bed with adjustable arm supports in the form of bars or rails extending alongside it. One or more robotic arms 39 (e.g., via a shoulder with an elbow joint) can be attached to the adjustable arm supports, which can be vertically adjusted. By providing vertical adjustment, the robotic arms 39 are advantageously capable of being stowed compactly beneath the patient table or bed, and subsequently raised during a procedure.

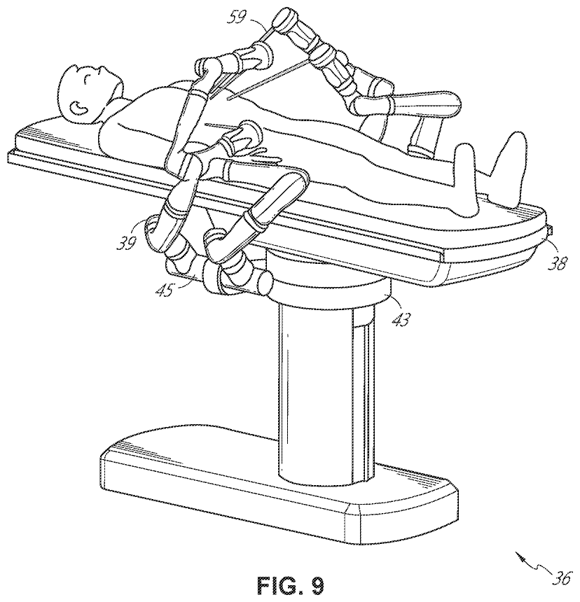

The arms 39 may be mounted on the carriages through a set of arm mounts 45 comprising a series of joints that may individually rotate and/or telescopically extend to provide additional configurability to the robotic arms 39. Additionally, the arm mounts 45 may be positioned on the carriages 43 such that, when the carriages 43 are appropriately rotated, the arm mounts 45 may be positioned on either the same side of table 38 (as shown in FIG. 6), on opposite sides of table 38 (as shown in FIG. 9), or on adjacent sides of the table 38 (not shown).

The column 37 structurally provides support for the table 38, and a path for vertical translation of the carriages. Internally, the column 37 may be equipped with lead screws for guiding vertical translation of the carriages, and motors to mechanize the translation of said carriages based the lead screws. The column 37 may also convey power and control signals to the carriage 43 and robotic arms 39 mounted thereon.

The table base 46 serves a similar function as the cart base 15 in cart 11 shown in FIG. 2, housing heavier components to balance the table/bed 38, the column 37, the carriages 43, and the robotic arms 39. The table base 46 may also incorporate rigid casters to provide stability during procedures. Deployed from the bottom of the table base 46, the casters may extend in opposite directions on both sides of the base 46 and retract when the system 36 needs to be moved.

Continuing with FIG. 6, the system 36 may also include a tower (not shown) that divides the functionality of system 36 between table and tower to reduce the form factor and bulk of the table. As in earlier disclosed embodiments, the tower may provide a variety of support functionalities to table, such as processing, computing, and control capabilities, power, fluidics, and/or optical and sensor processing. The tower may also be movable to be positioned away from the patient to improve physician access and de-clutter the operating room. Additionally, placing components in the tower allows for more storage space in the table base for potential stowage of the robotic arms. The tower may also include a master controller or console that provides both a user interface for user input, such as keyboard and/or pendant, as well as a display screen (or touchscreen) for pre-operative and intra-operative information, such as real-time imaging, navigation, and tracking information. In some embodiments, the tower may also contain holders for gas tanks to be used for insufflation.

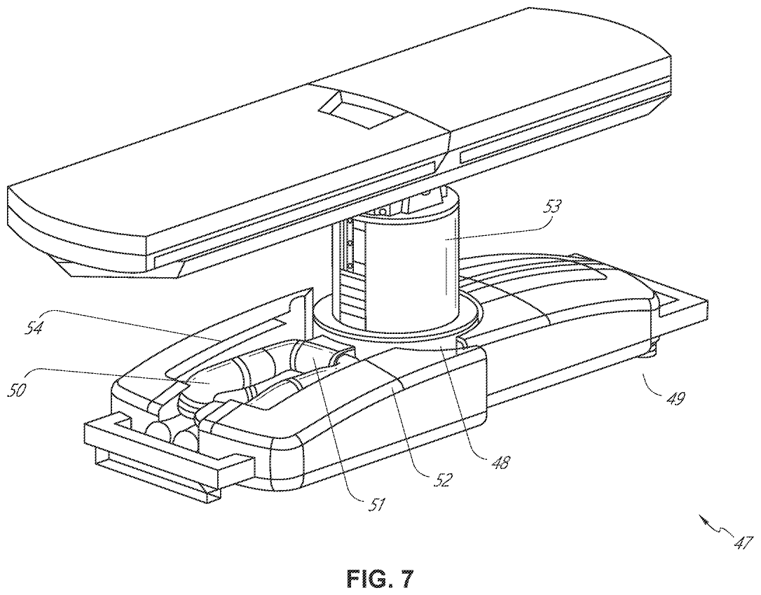

In some embodiments, a table base may stow and store the robotic arms when not in use. FIG. 7 illustrates a system 47 that stows robotic arms in an embodiment of the table-based system. In system 47, carriages 48 may be vertically translated into base 49 to stow robotic arms 50, arm mounts 51, and the carriages 48 within the base 49. Base covers 52 may be translated and retracted open to deploy the carriages 48, arm mounts 51, and arms 50 around column 53, and closed to stow to protect them when not in use. The base covers 52 may be sealed with a membrane 54 along the edges of its opening to prevent dirt and fluid ingress when closed.

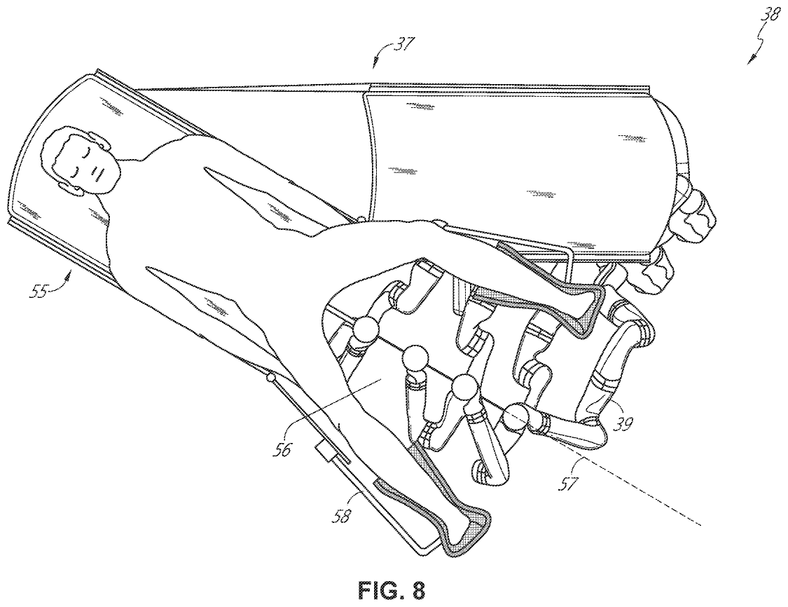

FIG. 8 illustrates an embodiment of a robotically-enabled table-based system configured for a ureteroscopy procedure. In a ureteroscopy, the table 38 may include a swivel portion 55 for positioning a patient off-angle from the column 37 and table base 46. The swivel portion 55 may rotate or pivot around a pivot point (e.g., located below the patient's head) in order to position the bottom portion of the swivel portion 55 away from the column 37. For example, the pivoting of the swivel portion 55 allows a C-arm (not shown) to be positioned over the patient's lower abdomen without competing for space with the column (not shown) below table 38. By rotating the carriage (not shown) around the column 37, the robotic arms 39 may directly insert a ureteroscope 56 along a virtual rail 57 into the patient's groin area to reach the urethra. In a ureteroscopy, stirrups 58 may also be fixed to the swivel portion 55 of the table 38 to support the position of the patient's legs during the procedure and allow clear access to the patient's groin area.

In a laparoscopic procedure, through small incision(s) in the patient's abdominal wall, minimally invasive instruments may be inserted into the patient's anatomy. In some embodiments, the minimally invasive instruments comprise an elongated rigid member, such as a shaft, which is used to access anatomy within the patient. After inflation of the patient's abdominal cavity, the instruments may be directed to perform surgical or medical tasks, such as grasping, cutting, ablating, suturing, etc. In some embodiments, the instruments can comprise a scope, such as a laparoscope. FIG. 9 illustrates an embodiment of a robotically-enabled table-based system configured for a laparoscopic procedure. As shown in FIG. 9, the carriages 43 of the system 36 may be rotated and vertically adjusted to position pairs of the robotic arms 39 on opposite sides of the table 38, such that instrument 59 may be positioned using the arm mounts 45 to be passed through minimal incisions on both sides of the patient to reach his/her abdominal cavity.

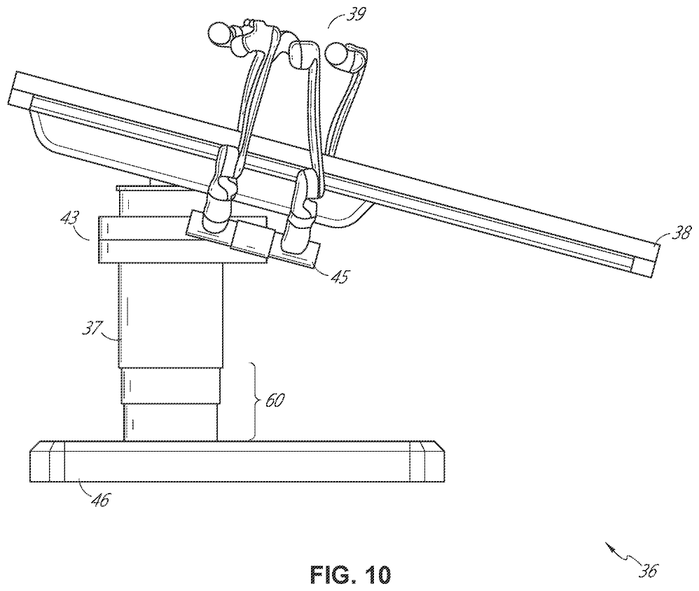

To accommodate laparoscopic procedures, the robotically-enabled table system may also tilt the platform to a desired angle. FIG. 10 illustrates an embodiment of the robotically-enabled medical system with pitch or tilt adjustment. As shown in FIG. 10, the system 36 may accommodate tilt of the table 38 to position one portion of the table at a greater distance from the floor than the other. Additionally, the arm mounts 45 may rotate to match the tilt such that the arms 39 maintain the same planar relationship with table 38. To accommodate steeper angles, the column 37 may also include telescoping portions 60 that allow vertical extension of column 37 to keep the table 38 from touching the floor or colliding with base 46.

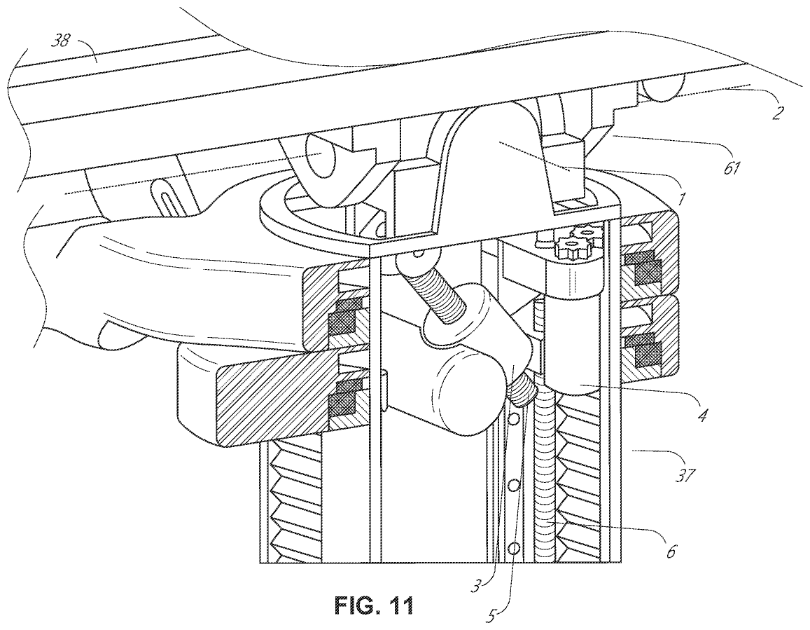

FIG. 11 provides a detailed illustration of the interface between the table 38 and the column 37. Pitch rotation mechanism 61 may be configured to alter the pitch angle of the table 38 relative to the column 37 in multiple degrees of freedom. The pitch rotation mechanism 61 may be enabled by the positioning of orthogonal axes 1, 2 at the column-table interface, each axis actuated by a separate motor 3, 4 responsive to an electrical pitch angle command. Rotation along one screw 5 would enable tilt adjustments in one axis 1, while rotation along the other screw 6 would enable tilt adjustments along the other axis 2. In some embodiments, a ball joint can be used to alter the pitch angle of the table 38 relative to the column 37 in multiple degrees of freedom.

For example, pitch adjustments are particularly useful when trying to position the table in a Trendelenburg position, i.e., position the patient's lower abdomen at a higher position from the floor than the patient's lower abdomen, for lower abdominal surgery. The Trendelenburg position causes the patient's internal organs to slide towards his/her upper abdomen through the force of gravity, clearing out the abdominal cavity for minimally invasive tools to enter and perform lower abdominal surgical or medical procedures, such as laparoscopic prostatectomy.

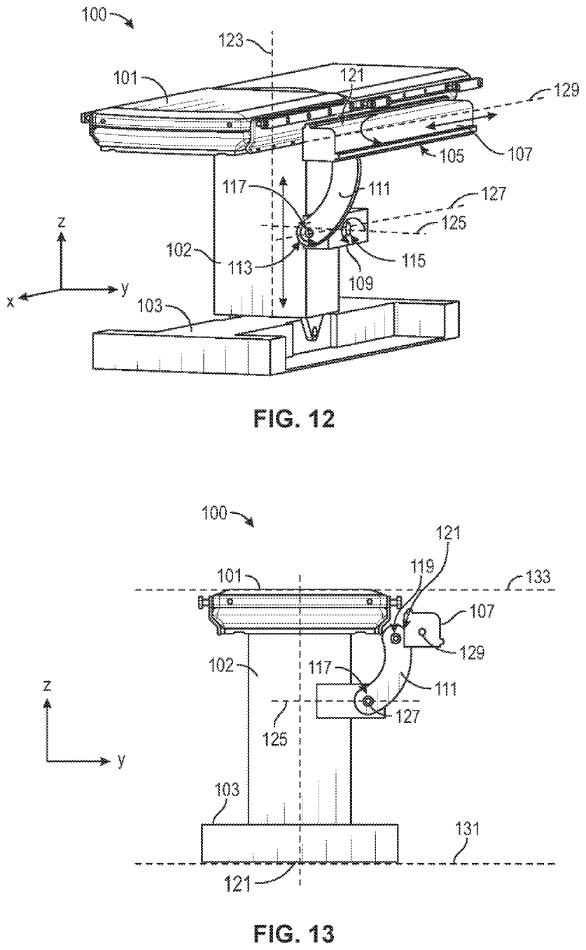

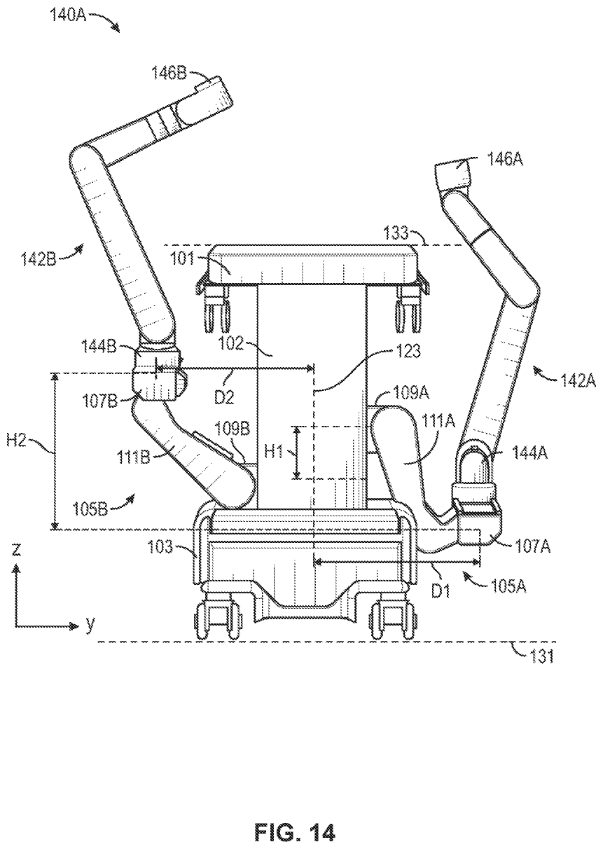

FIGS. 12 and 13 illustrate isometric and end views of an alternative embodiment of a table-based surgical robotics system 100. The surgical robotics system 100 includes one or more adjustable arm supports 105 that can be configured to support one or more robotic arms (see, for example, FIG. 14) relative to a table 101. In the illustrated embodiment, a single adjustable arm support 105 is shown, though an additional arm support can be provided on an opposite side of the table 101. The adjustable arm support 105 can be configured so that it can move relative to the table 101 to adjust and/or vary the position of the adjustable arm support 105 and/or any robotic arms mounted thereto relative to the table 101. For example, the adjustable arm support 105 may be adjusted one or more degrees of freedom relative to the table 101. The adjustable arm support 105 provides high versatility to the system 100, including the ability to easily stow the one or more adjustable arm supports 105 and any robotics arms attached thereto beneath the table 101. The adjustable arm support 105 can be elevated from the stowed position to a position below an upper surface of the table 101. In other embodiments, the adjustable arm support 105 can be elevated from the stowed position to a position above an upper surface of the table 101.

The adjustable arm support 105 can provide several degrees of freedom, including lift, lateral translation, tilt, etc. In the illustrated embodiment of FIGS. 12 and 13, the arm support 105 is configured with four degrees of freedom, which are illustrated with arrows in FIG. 12. A first degree of freedom allows for adjustment of the adjustable arm support 105 in the z-direction (“Z-lift”). For example, the adjustable arm support 105 can include a carriage 109 configured to move up or down along or relative to a column 102 supporting the table 101. A second degree of freedom can allow the adjustable arm support 105 to tilt. For example, the adjustable arm support 105 can include a rotary joint, which can allow the adjustable arm support 105 to be aligned with the bed in a Trendelenburg position. A third degree of freedom can allow the adjustable arm support 105 to “pivot up,” which can be used to adjust a distance between a side of the table 101 and the adjustable arm support 105. A fourth degree of freedom can permit translation of the adjustable arm support 105 along a longitudinal length of the table.

The surgical robotics system 100 in FIGS. 12 and 13 can comprise a table supported by a column 102 that is mounted to a base 103. The base 103 and the column 102 support the table 101 relative to a support surface. A floor axis 131 and a support axis 133 are shown in FIG. 13.

The adjustable arm support 105 can be mounted to the column 102. In other embodiments, the arm support 105 can be mounted to the table 101 or base 103. The adjustable arm support 105 can include a carriage 109, a bar or rail connector 111 and a bar or rail 107. In some embodiments, one or more robotic arms mounted to the rail 107 can translate and move relative to one another.

The carriage 109 can be attached to the column 102 by a first joint 113, which allows the carriage 109 to move relative to the column 102 (e.g., such as up and down a first or vertical axis 123). The first joint 113 can provide the first degree of freedom (“Z-lift”) to the adjustable arm support 105. The adjustable arm support 105 can include a second joint 115, which provides the second degree of freedom (tilt) for the adjustable arm support 105. The adjustable arm support 105 can include a third joint 117, which can provide the third degree of freedom (“pivot up”) for the adjustable arm support 105. An additional joint 119 (shown in FIG. 13) can be provided that mechanically constrains the third joint 117 to maintain an orientation of the rail 107 as the rail connector 111 is rotated about a third axis 127. The adjustable arm support 105 can include a fourth joint 121, which can provide a fourth degree of freedom (translation) for the adjustable arm support 105 along a fourth axis 129.

FIG. 14 illustrates an end view of the surgical robotics system 140A with two adjustable arm supports 105A, 105B mounted on opposite sides of a table 101. A first robotic arm 142A is attached to the bar or rail 107A of the first adjustable arm support 105B. The first robotic arm 142A includes a base 144A attached to the rail 107A. The distal end of the first robotic arm 142A includes an instrument drive mechanism 146A that can attach to one or more robotic medical instruments or tools. Similarly, the second robotic arm 142B includes a base 144B attached to the rail 107B. The distal end of the second robotic arm 142B includes an instrument drive mechanism 146B. The instrument drive mechanism 146B can be configured to attach to one or more robotic medical instruments or tools.

In some embodiments, one or more of the robotic arms 142A, 142B comprises an arm with seven or more degrees of freedom. In some embodiments, one or more of the robotic arms 142A, 142B can include eight degrees of freedom, including an insertion axis (1-degree of freedom including insertion), a wrist (3-degrees of freedom including wrist pitch, yaw and roll), an elbow (1-degree of freedom including elbow pitch), a shoulder (2-degrees of freedom including shoulder pitch and yaw), and base 144A, 144B (1-degree of freedom including translation). In some embodiments, the insertion degree of freedom can be provided by the robotic arm 142A, 142B, while in other embodiments, the instrument itself provides insertion via an instrument-based insertion architecture.

### C. Instrument Driver & Interface.

The end effectors of the system's robotic arms comprise (i) an instrument driver (alternatively referred to as “instrument drive mechanism” or “instrument device manipulator”) that incorporate electro-mechanical means for actuating the medical instrument and (ii) a removable or detachable medical instrument, which may be devoid of any electro-mechanical components, such as motors. This dichotomy may be driven by the need to sterilize medical instruments used in medical procedures, and the inability to adequately sterilize expensive capital equipment due to their intricate mechanical assemblies and sensitive electronics. Accordingly, the medical instruments may be designed to be detached, removed, and interchanged from the instrument driver (and thus the system) for individual sterilization or disposal by the physician or the physician's staff. In contrast, the instrument drivers need not be changed or sterilized, and may be draped for protection.

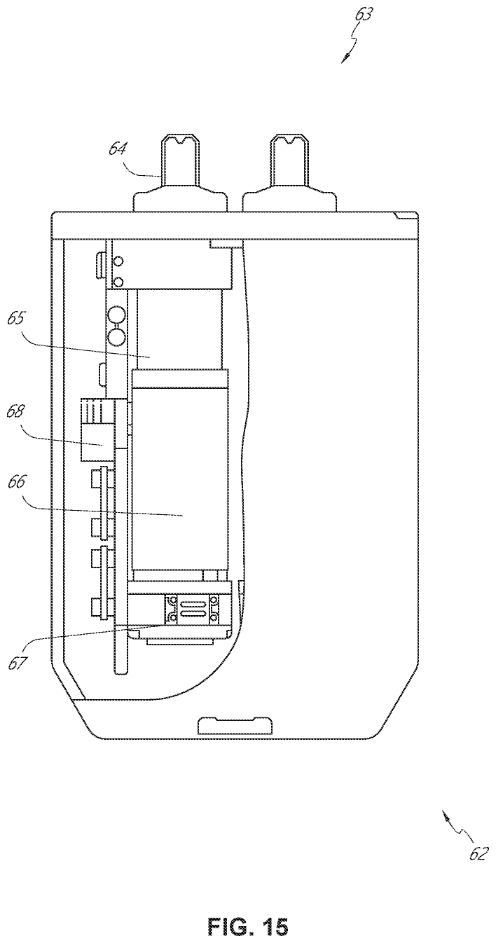

FIG. 15 illustrates an example instrument driver. Positioned at the distal end of a robotic arm, instrument driver 62 comprises of one or more drive units 63 arranged with parallel axes to provide controlled torque to a medical instrument via drive shafts 64. Each drive unit 63 comprises an individual drive shaft 64 for interacting with the instrument, a gear head 65 for converting the motor shaft rotation to a desired torque, a motor 66 for generating the drive torque, an encoder 67 to measure the speed of the motor shaft and provide feedback to the control circuitry, and control circuity 68 for receiving control signals and actuating the drive unit. Each drive unit 63 being independent controlled and motorized, the instrument driver 62 may provide multiple (four as shown in FIG. 15) independent drive outputs to the medical instrument. In operation, the control circuitry 68 would receive a control signal, transmit a motor signal to the motor 66, compare the resulting motor speed as measured by the encoder 67 with the desired speed, and modulate the motor signal to generate the desired torque.

For procedures that require a sterile environment, the robotic system may incorporate a drive interface, such as a sterile adapter connected to a sterile drape, that sits between the instrument driver and the medical instrument. The chief purpose of the sterile adapter is to transfer angular motion from the drive shafts of the instrument driver to the drive inputs of the instrument while maintaining physical separation, and thus sterility, between the drive shafts and drive inputs. Accordingly, an example sterile adapter may comprise of a series of rotational inputs and outputs intended to be mated with the drive shafts of the instrument driver and drive inputs on the instrument. Connected to the sterile adapter, the sterile drape, comprised of a thin, flexible material such as transparent or translucent plastic, is designed to cover the capital equipment, such as the instrument driver, robotic arm, and cart (in a cart-based system) or table (in a table-based system). Use of the drape would allow the capital equipment to be positioned proximate to the patient while still being located in an area not requiring sterilization (i.e., non-sterile field). On the other side of the sterile drape, the medical instrument may interface with the patient in an area requiring sterilization (i.e., sterile field).

### D. Medical Instrument.

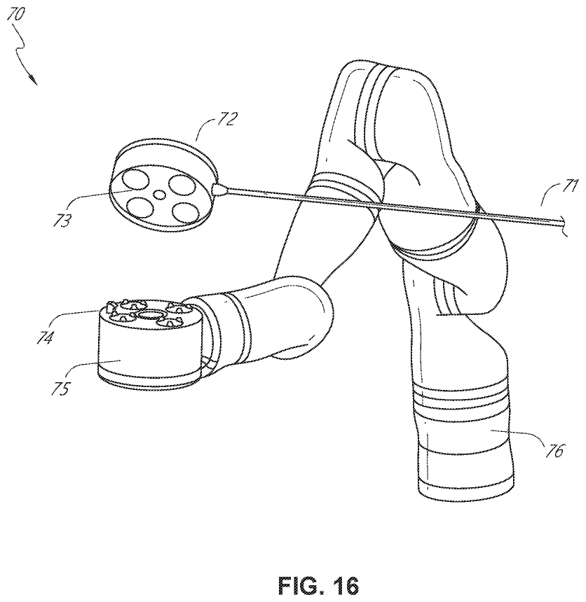

FIG. 16 illustrates an example medical instrument with a paired instrument driver. Like other instruments designed for use with a robotic system, medical instrument 70 comprises an elongated shaft 71 (or elongate body) and an instrument base 72. The instrument base 72, also referred to as an “instrument handle” due to its intended design for manual interaction by the physician, may generally comprise rotatable drive inputs 73, e.g., receptacles, pulleys or spools, that are designed to be mated with drive outputs 74 that extend through a drive interface on instrument driver 75 at the distal end of robotic arm 76. When physically connected, latched, and/or coupled, the mated drive inputs 73 of instrument base 72 may share axes of rotation with the drive outputs 74 in the instrument driver 75 to allow the transfer of torque from drive outputs 74 to drive inputs 73. In some embodiments, the drive outputs 74 may comprise splines that are designed to mate with receptacles on the drive inputs 73.

The elongated shaft 71 is designed to be delivered through either an anatomical opening or lumen, e.g., as in endoscopy, or a minimally invasive incision, e.g., as in laparoscopy. The elongated shaft 71 may be either flexible (e.g., having properties similar to an endoscope) or rigid (e.g., having properties similar to a laparoscope) or contain a customized combination of both flexible and rigid portions. When designed for laparoscopy, the distal end of a rigid elongated shaft may be connected to an end effector extending from a jointed wrist formed from a clevis with at least one degree of freedom and a surgical tool or medical instrument, such as, for example, a grasper or scissors, that may be actuated based on force from the tendons as the drive inputs rotate in response to torque received from the drive outputs 74 of the instrument driver 75. When designed for endoscopy, the distal end of a flexible elongated shaft may include a steerable or controllable bending section that may be articulated and bent based on torque received from the drive outputs 74 of the instrument driver 75.

Torque from the instrument driver 75 is transmitted down the elongated shaft 71 using tendons along the shaft 71. These individual tendons, such as pull wires, may be individually anchored to individual drive inputs 73 within the instrument handle 72. From the handle 72, the tendons are directed down one or more pull lumens along the elongated shaft 71 and anchored at the distal portion of the elongated shaft 71, or in the wrist at the distal portion of the elongated shaft. During a surgical procedure, such as a laparoscopic, endoscopic or hybrid procedure, these tendons may be coupled to a distally mounted end effector, such as a wrist, grasper, or scissor. Under such an arrangement, torque exerted on drive inputs 73 would transfer tension to the tendon, thereby causing the end effector to actuate in some way. In some embodiments, during a surgical procedure, the tendon may cause a joint to rotate about an axis, thereby causing the end effector to move in one direction or another. Alternatively, the tendon may be connected to one or more jaws of a grasper at distal end of the elongated shaft 71, where tension from the tendon cause the grasper to close.

In endoscopy, the tendons may be coupled to a bending or articulating section positioned along the elongated shaft 71 (e.g., at the distal end) via adhesive, control ring, or other mechanical fixation. When fixedly attached to the distal end of a bending section, torque exerted on drive inputs 73 would be transmitted down the tendons, causing the softer, bending section (sometimes referred to as the articulable section or region) to bend or articulate. Along the non-bending sections, it may be advantageous to spiral or helix the individual pull lumens that direct the individual tendons along (or inside) the walls of the endoscope shaft to balance the radial forces that result from tension in the pull wires. The angle of the spiraling and/or spacing there between may be altered or engineered for specific purposes, wherein tighter spiraling exhibits lesser shaft compression under load forces, while lower amounts of spiraling results in greater shaft compression under load forces, but also exhibits limits bending. On the other end of the spectrum, the pull lumens may be directed parallel to the longitudinal axis of the elongated shaft 71 to allow for controlled articulation in the desired bending or articulable sections.

In endoscopy, the elongated shaft 71 houses a number of components to assist with the robotic procedure. The shaft may comprise of a working channel for deploying surgical tools (or medical instruments), irrigation, and/or aspiration to the operative region at the distal end of the shaft 71. The shaft 71 may also accommodate wires and/or optical fibers to transfer signals to/from an optical assembly at the distal tip, which may include of an optical camera. The shaft 71 may also accommodate optical fibers to carry light from proximally-located light sources, such as light emitting diodes, to the distal end of the shaft.

At the distal end of the instrument 70, the distal tip may also comprise the opening of a working channel for delivering tools for diagnostic and/or therapy, irrigation, and aspiration to an operative site. The distal tip may also include a port for a camera, such as a fiberscope or a digital camera, to capture images of an internal anatomical space. Relatedly, the distal tip may also include ports for light sources for illuminating the anatomical space when using the camera.

In the example of FIG. 16, the drive shaft axes, and thus the drive input axes, are orthogonal to the axis of the elongated shaft. This arrangement, however, complicates roll capabilities for the elongated shaft 71. Rolling the elongated shaft 71 along its axis while keeping the drive inputs 73 static results in undesirable tangling of the tendons as they extend off the drive inputs 73 and enter pull lumens within the elongated shaft 71. The resulting entanglement of such tendons may disrupt any control algorithms intended to predict movement of the flexible elongated shaft during an endoscopic procedure.

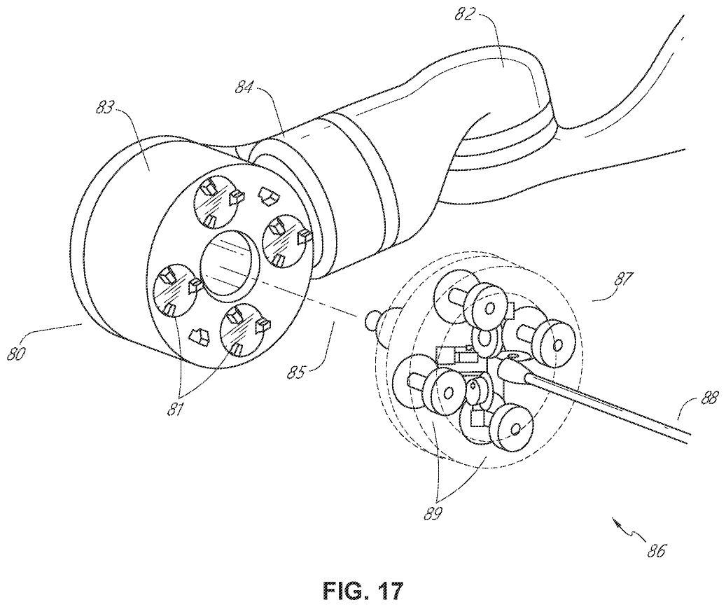

FIG. 17 illustrates an alternative design for an instrument driver and instrument where the axes of the drive units are parallel to the axis of the elongated shaft of the instrument. As shown, a circular instrument driver 80 comprises four drive units with their drive outputs 81 aligned in parallel at the end of a robotic arm 82. The drive units, and their respective drive outputs 81, are housed in a rotational assembly 83 of the instrument driver 80 that is driven by one of the drive units within the assembly 83. In response to torque provided by the rotational drive unit, the rotational assembly 83 rotates along a circular bearing that connects the rotational assembly 83 to the non-rotational portion 84 of the instrument driver. Power and controls signals may be communicated from the non-rotational portion 84 of the instrument driver 80 to the rotational assembly 83 through electrical contacts may be maintained through rotation by a brushed slip ring connection (not shown). In other embodiments, the rotational assembly 83 may be responsive to a separate drive unit that is integrated into the non-rotatable portion 84, and thus not in parallel to the other drive units. The rotational mechanism 83 allows the instrument driver 80 to rotate the drive units, and their respective drive outputs 81, as a single unit around an instrument driver axis 85.

Like earlier disclosed embodiments, an instrument 86 may comprise an elongated shaft portion 88 and an instrument base 87 (shown with a transparent external skin for discussion purposes) comprising a plurality of drive inputs 89 (such as receptacles, pulleys, and spools) that are configured to receive the drive outputs 81 in the instrument driver 80. Unlike prior disclosed embodiments, instrument shaft 88 extends from the center of instrument base 87 with an axis substantially parallel to the axes of the drive inputs 89, rather than orthogonal as in the design of FIG. 16.

When coupled to the rotational assembly 83 of the instrument driver 80, the medical instrument 86, comprising instrument base 87 and instrument shaft 88, rotates in combination with the rotational assembly 83 about the instrument driver axis 85. Since the instrument shaft 88 is positioned at the center of instrument base 87, the instrument shaft 88 is coaxial with instrument driver axis 85 when attached. Thus, rotation of the rotational assembly 83 causes the instrument shaft 88 to rotate about its own longitudinal axis. Moreover, as the instrument base 87 rotates with the instrument shaft 88, any tendons connected to the drive inputs 89 in the instrument base 87 are not tangled during rotation. Accordingly, the parallelism of the axes of the drive outputs 81, drive inputs 89, and instrument shaft 88 allows for the shaft rotation without tangling any control tendons.

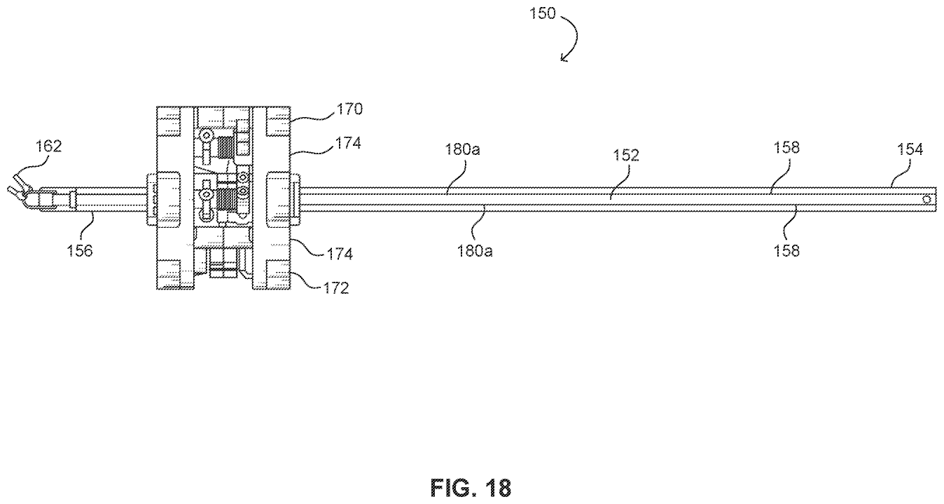

FIG. 18 illustrates an instrument having an instrument based insertion architecture in accordance with some embodiments. The instrument 150 can be coupled to any of the instrument drivers discussed above. The instrument 150 comprises an elongated shaft 152, an end effector 162 connected to the shaft 152, and a handle 170 coupled to the shaft 152. The elongated shaft 152 comprises a tubular member having a proximal portion 154 and a distal portion 156. The elongated shaft 152 comprises one or more channels or grooves 158 along its outer surface. The grooves 158 are configured to receive one or more wires or cables 180 therethrough. One or more cables 180 thus run along an outer surface of the elongated shaft 152. In other embodiments, cables 180 can also run through the elongated shaft 152. Manipulation of the one or more cables 180 (e.g., via an instrument driver) results in actuation of the end effector 162.

The instrument handle 170, which may also be referred to as an instrument base, may generally comprise an attachment interface 172 having one or more mechanical inputs 174, e.g., receptacles, pulleys or spools, that are designed to be reciprocally mated with one or more torque couplers on an attachment surface of an instrument driver.

In some embodiments, the instrument 150 comprises a series of pulleys or cables that enable the elongated shaft 152 to translate relative to the handle 170. In other words, the instrument 150 itself comprises an instrument-based insertion architecture that accommodates insertion of the instrument, thereby minimizing the reliance on a robot arm to provide insertion of the instrument 150. In other embodiments, a robotic arm can be largely responsible for instrument insertion.

### E. Controller.

Any of the robotic systems described herein can include an input device or controller for manipulating an instrument attached to a robotic arm. In some embodiments, the controller can be coupled (e.g., communicatively, electronically, electrically, wirelessly and/or mechanically) with an instrument such that manipulation of the controller causes a corresponding manipulation of the instrument e.g., via master slave control.

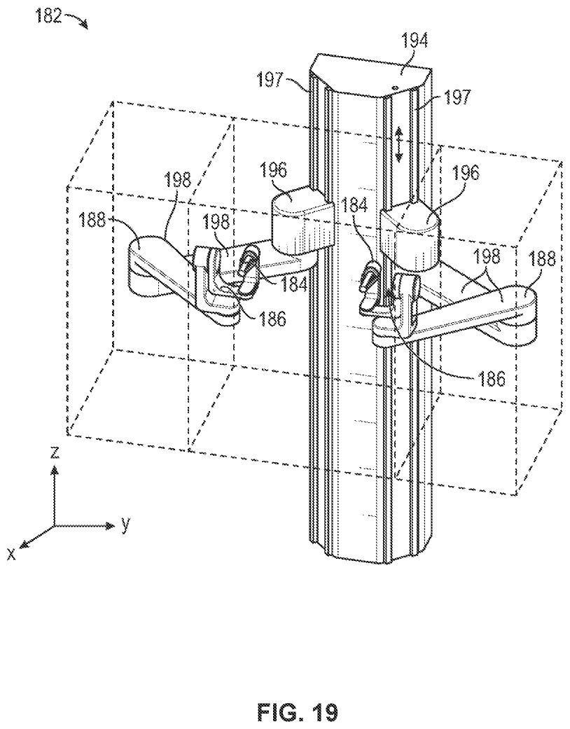

FIG. 19 is a perspective view of an embodiment of a controller 182. In the present embodiment, the controller 182 comprises a hybrid controller that can have both impedance and admittance control. In other embodiments, the controller 182 can utilize just impedance or passive control. In other embodiments, the controller 182 can utilize just admittance control. By being a hybrid controller, the controller 182 advantageously can have a lower perceived inertia while in use.

In the illustrated embodiment, the controller 182 is configured to allow manipulation of two medical instruments, and includes two handles 184. Each of the handles 184 is connected to a gimbal 186. Each gimbal 186 is connected to a positioning platform 188.

As shown in FIG. 19, each positioning platform 188 includes a SCARA arm (selective compliance assembly robot arm) 198 coupled to a column 194 by a prismatic joint 196. The prismatic joints 196 are configured to translate along the column 194 (e.g., along rails 197) to allow each of the handles 184 to be translated in the z-direction, providing a first degree of freedom. The SCARA arm 198 is configured to allow motion of the handle 184 in an x-y plane, providing two additional degrees of freedom.

In some embodiments, one or more load cells are positioned in the controller. For example, in some embodiments, a load cell (not shown) is positioned in the body of each of the gimbals 186. By providing a load cell, portions of the controller 182 are capable of operating under admittance control, thereby advantageously reducing the perceived inertia of the controller while in use. In some embodiments, the positioning platform 188 is configured for admittance control, while the gimbal 186 is configured for impedance control. In other embodiments, the gimbal 186 is configured for admittance control, while the positioning platform 188 is configured for impedance control. Accordingly, for some embodiments, the translational or positional degrees of freedom of the positioning platform 188 can rely on admittance control, while the rotational degrees of freedom of the gimbal 186 rely on impedance control.

### F. Navigation and Control.

Traditional endoscopy may involve the use of fluoroscopy (e.g., as may be delivered through a C-arm) and other forms of radiation-based imaging modalities to provide endoluminal guidance to an operator physician. In contrast, the robotic systems contemplated by this disclosure can provide for non-radiation-based navigational and localization means to reduce physician exposure to radiation and reduce the amount of equipment within the operating room. As used herein, the term “localization” may refer to determining and/or monitoring the position of objects in a reference coordinate system. Technologies such as pre-operative mapping, computer vision, real-time EM tracking, and robot command data may be used individually or in combination to achieve a radiation-free operating environment. In other cases, where radiation-based imaging modalities are still used, the pre-operative mapping, computer vision, real-time EM tracking, and robot command data may be used individually or in combination to improve upon the information obtained solely through radiation-based imaging modalities.

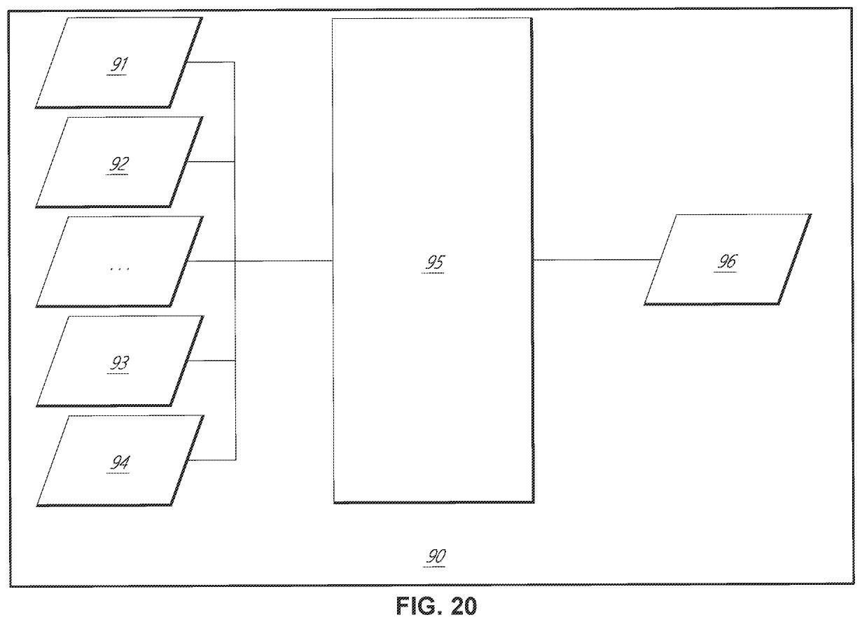

FIG. 20 is a block diagram illustrating a localization system 90 that estimates a location of one or more elements of the robotic system, such as the location of the instrument, in accordance to an example embodiment. The localization system 90 may be a set of one or more computer devices configured to execute one or more instructions. The computer devices may be embodied by a processor (or processors) and computer-readable memory in one or more components discussed above. By way of example and not limitation, the computer devices may be in the tower 30 shown in FIG. 1, the cart shown in FIGS. 1-4, the beds shown in FIGS. 5-14, etc.

As shown in FIG. 20, the localization system 90 may include a localization module 95 that processes input data 91-94 to generate location data 96 for the distal tip of a medical instrument. The location data 96 may be data or logic that represents a location and/or orientation of the distal end of the instrument relative to a frame of reference. The frame of reference can be a frame of reference relative to the anatomy of the patient or to a known object, such as an EM field generator (see discussion below for the EM field generator).

The various input data 91-94 are now described in greater detail. Pre-operative mapping may be accomplished through the use of the collection of low dose CT scans. Pre-operative CT scans are reconstructed into three-dimensional images, which are visualized, e.g. as “slices” of a cutaway view of the patient's internal anatomy. When analyzed in the aggregate, image-based models for anatomical cavities, spaces and structures of the patient's anatomy, such as a patient lung network, may be generated. Techniques such as center-line geometry may be determined and approximated from the CT images to develop a three-dimensional volume of the patient's anatomy, referred to as model data 91 (also referred to as “preoperative model data” when generated using only preoperative CT scans). The use of center-line geometry is discussed in U.S. patent application Ser. No. 14/523,760, the contents of which are herein incorporated in its entirety. Network topological models may also be derived from the CT-images, and are particularly appropriate for bronchoscopy.

In some embodiments, the instrument may be equipped with a camera to provide vision data 92. The localization module 95 may process the vision data to enable one or more vision-based location tracking. For example, the preoperative model data may be used in conjunction with the vision data 92 to enable computer vision-based tracking of the medical instrument (e.g., an endoscope or an instrument advance through a working channel of the endoscope). For example, using the preoperative model data 91, the robotic system may generate a library of expected endoscopic images from the model based on the expected path of travel of the endoscope, each image linked to a location within the model. Intra-operatively, this library may be referenced by the robotic system in order to compare real-time images captured at the camera (e.g., a camera at a distal end of the endoscope) to those in the image library to assist localization.

Other computer vision-based tracking techniques use feature tracking to determine motion of the camera, and thus the endoscope. Some features of the localization module 95 may identify circular geometries in the preoperative model data 91 that correspond to anatomical lumens and track the change of those geometries to determine which anatomical lumen was selected, as well as the relative rotational and/or translational motion of the camera. Use of a topological map may further enhance vision-based algorithms or techniques.

Optical flow, another computer vision-based technique, may analyze the displacement and translation of image pixels in a video sequence in the vision data 92 to infer camera movement. Examples of optical flow techniques may include motion detection, object segmentation calculations, luminance, motion compensated encoding, stereo disparity measurement, etc. Through the comparison of multiple frames over multiple iterations, movement and location of the camera (and thus the endoscope) may be determined.

The localization module 95 may use real-time EM tracking to generate a real-time location of the endoscope in a global coordinate system that may be registered to the patient's anatomy, represented by the preoperative model. In EM tracking, an EM sensor (or tracker) comprising of one or more sensor coils embedded in one or more locations and orientations in a medical instrument (e.g., an endoscopic tool) measures the variation in the EM field created by one or more static EM field generators positioned at a known location. The location information detected by the EM sensors is stored as EM data 93. The EM field generator (or transmitter), may be placed close to the patient to create a low intensity magnetic field that the embedded sensor may detect. The magnetic field induces small currents in the sensor coils of the EM sensor, which may be analyzed to determine the distance and angle between the EM sensor and the EM field generator. These distances and orientations may be intra-operatively “registered” to the patient anatomy (e.g., the preoperative model) in order to determine the geometric transformation that aligns a single location in the coordinate system with a position in the pre-operative model of the patient's anatomy. Once registered, an embedded EM tracker in one or more positions of the medical instrument (e.g., the distal tip of an endoscope) may provide real-time indications of the progression of the medical instrument through the patient's anatomy.

Robotic command and kinematics data 94 may also be used by the localization module 95 to provide localization data 96 for the robotic system. Device pitch and yaw resulting from articulation commands may be determined during pre-operative calibration. Intra-operatively, these calibration measurements may be used in combination with known insertion depth information to estimate the position of the instrument. Alternatively, these calculations may be analyzed in combination with EM, vision, and/or topological modeling to estimate the position of the medical instrument within the network.

As FIG. 20 shows, a number of other input data can be used by the localization module 95. For example, although not shown in FIG. 20, an instrument utilizing shape-sensing fiber can provide shape data that the localization module 95 can use to determine the location and shape of the instrument.

The localization module 95 may use the input data 91-94 in combination(s). In some cases, such a combination may use a probabilistic approach where the localization module 95 assigns a confidence weight to the location determined from each of the input data 91-94. Thus, where the EM data may not be reliable (as may be the case where there is EM interference) the confidence of the location determined by the EM data 93 can be decrease and the localization module 95 may rely more heavily on the vision data 92 and/or the robotic command and kinematics data 94.

As discussed above, the robotic systems discussed herein may be designed to incorporate a combination of one or more of the technologies above. The robotic system's computer-based control system, based in the tower, bed and/or cart, may store computer program instructions, for example, within a non-transitory computer-readable storage medium such as a persistent magnetic storage drive, solid state drive, or the like, that, upon execution, cause the system to receive and analyze sensor data and user commands, generate control signals throughout the system, and display the navigational and localization data, such as the position of the instrument within the global coordinate system, anatomical map, etc.

### 2. Wheel Assemblies for Power Assisted Mobile Medical Platforms.

As shown in several of the examples described above, robotic medical systems can include a medical platform that includes a bed or table top. The medical platform can be configured to support a patient during a medical procedure, such as robotic endoscopy, robotic laparoscopy, open procedures, or others (see, for example, FIGS. 1, 3, 4, 5, 8, and 9, described above). In some cases, the medical platform may need to be moved, and the use of motorized wheels for power-assisted mobility can provide ease of maneuverability and precision of movement to the medical platform. Due to the heavy weight of the bed, and in particular, a bed with one or more robotic arms coupled thereto, it can be a challenge to propel and maneuver the bed.

Disclosed herein are mobile medical platforms that utilize one or more wheel assemblies (also referred to herein as powered wheel assemblies or motorized wheel assemblies) to provide power-assisted mobility. A wheel assembly includes a wheel that is powered by one or more motors, advantageously allowing motorized control over both steering and propulsion of the wheel. Such configuration facilitates accurate positioning of mobile medical platforms and allows maneuvers that were difficult to perform with conventional beds. In some embodiments, the wheel has a negligible caster angle, which facilitates the wheel to be simultaneously steered and rolled.

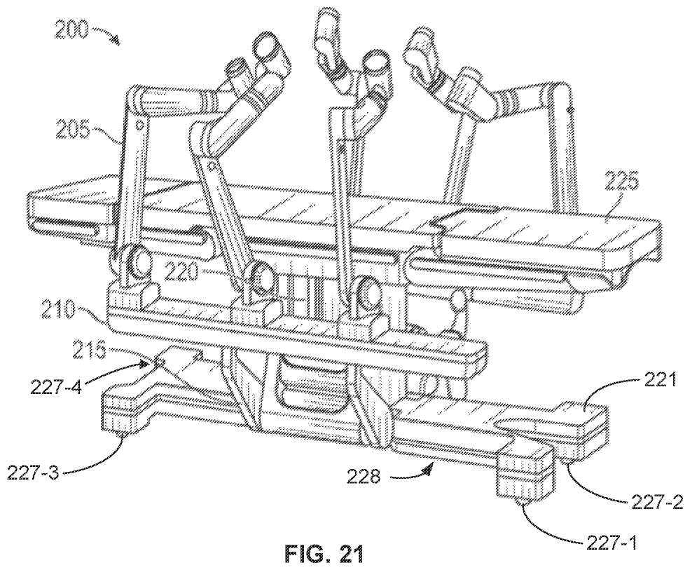

FIG. 21 illustrates a mobile medical platform 200 in accordance with some embodiments. The mobile medical platform 200 (e.g., patient platform, robotic surgical platform) includes a rigid base 221 and one or more powered wheel assemblies 227 (e.g., 227-1 through 227-4). The one or more power wheel assemblies 227 are coupled (e.g., rigidly coupled) to a first side 228 (e.g., a bottom side) of the rigid base 221 and configured to provide power-assisted movement and transportation of the entire mobile medical platform 200.

In some embodiments, the mobile medical platform 200 also includes a table top 225 (e.g., surgical bed, surgical table, robotic surgical table) and a bed column 220 to support the table top 225. The table top 225 is configured to support a patient and serve as a hospital bed or a surgical bed. The rigid base 221 (e.g., a table base for a surgical bed, a rigid load-bearing housing, a chassis, etc.) is configured to support the table top 225 (e.g., with a bed column 220).

In some embodiments, the mobile medical platform 200 also includes a plurality of robotic arms 205, one or more adjustable arm supports 210, and one or more set-up joints 215. Each of the robotic arms 205 may be supported by one of the adjustable arm supports 210 and the adjustable arm support(s) 210 may be in turn supported by the set-up joint(s) 215. In some embodiments, the mobile medical platform 200 includes medical equipment such as monitoring or imaging equipment attached to the one or more robotic arms 205. The mobile medical platform 200 may also include an onboard battery for wireless operation of the mobile medical platform 200 and/or an onboard power supply that can be plugged into an electrical socket to provide electricity for operation of the mobile medical platform 200. The one or more power wheel assemblies 227 are configured to provide power-assisted mobility to the mobile medical platform 200 and any equipment or persons that are support by, mounted on, coupled to, and/or on onboard, the mobile medical platform 200.

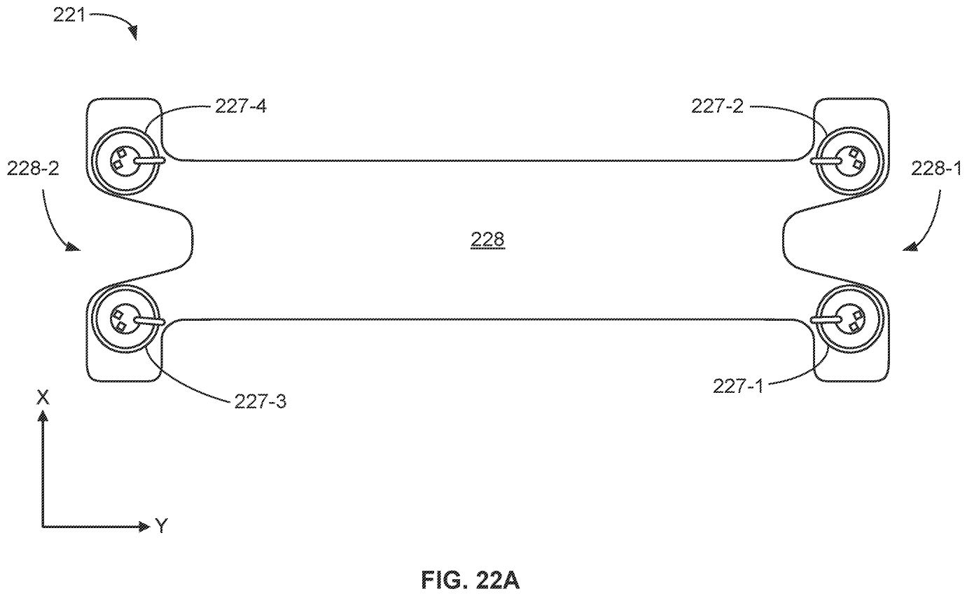

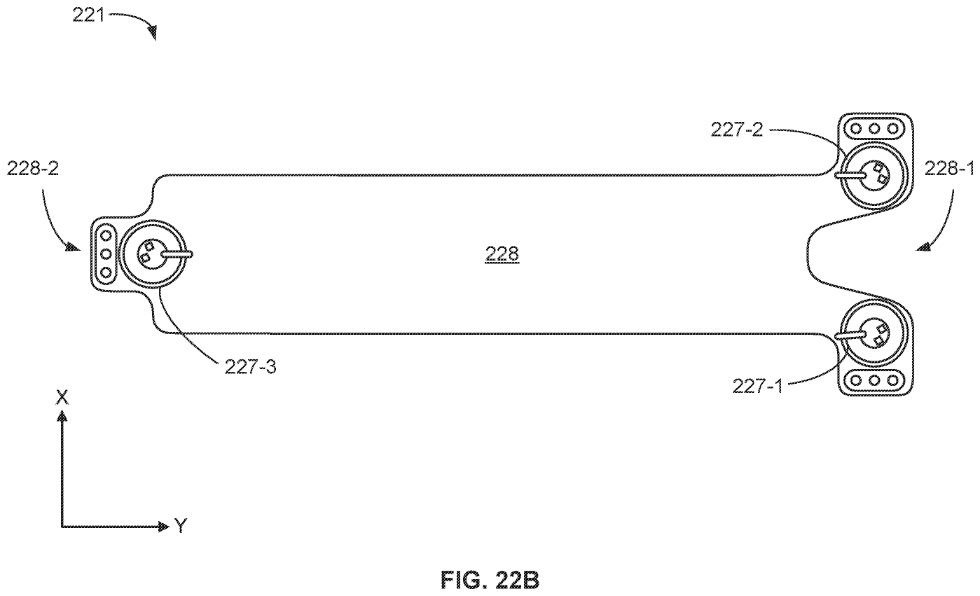

Examples of mobile medical platforms 200 that include one or more wheel assemblies 227 are shown with respect to FIGS. 22A and 22B.

FIG. 22A illustrates a bottom view of the mobile medical platform 200 of FIG. 21 having four wheel assemblies, 227-1 through 227-4 in accordance with some embodiments. In FIG. 22A, two wheel assemblies 227-1 and 227-2 are disposed toward a front end 228-1 of the rigid base 221 and two wheel assemblies 227-3 and 227-4 are disposed toward a back end 228-2 of the rigid base 221.

FIG. 22B illustrates a bottom view of a mobile medical platform 200 having three wheel assemblies, 227-1 through 227-3. Two wheel assemblies, 227-1 and 227-2, are disposed toward a front end 228-1 of the rigid base, and a wheel assembly 227-3 is disposed toward a back end 228-2 of the rigid base 221.

While FIGS. 22A and 22B illustrate mobile medical platforms 200 that include four powered wheel assemblies 227 and three powered wheel assemblies 227, respectively, a mobile medical platform 200 may have any number (greater than zero) of powered wheel assemblies 227. For example, a mobile medical platform 200 may have one, two, three, four, or more powered wheel assemblies 227. Further, the powered wheel assemblies 227 may be used to support and provide mobility to a mobile medical platform 200 in conjunction with a conventional wheel. For example, wheel assemblies 227-1 and 227-2 of the mobile medical platform 200 shown in FIG. 22B may be replaced by conventional wheels, and the powered wheel assembly 227-3 can provide power-assisted mobility for the mobile medical platform 200. When the mobile medical platform 200 includes at least one powered wheel assembly 227, the powered wheel assembly 227 is able to provide the mobile medical platform 200 with power-assisted mobility regardless of whether the mobile medical platform 200 includes additional powered wheel assemblies 227 or conventional wheels.

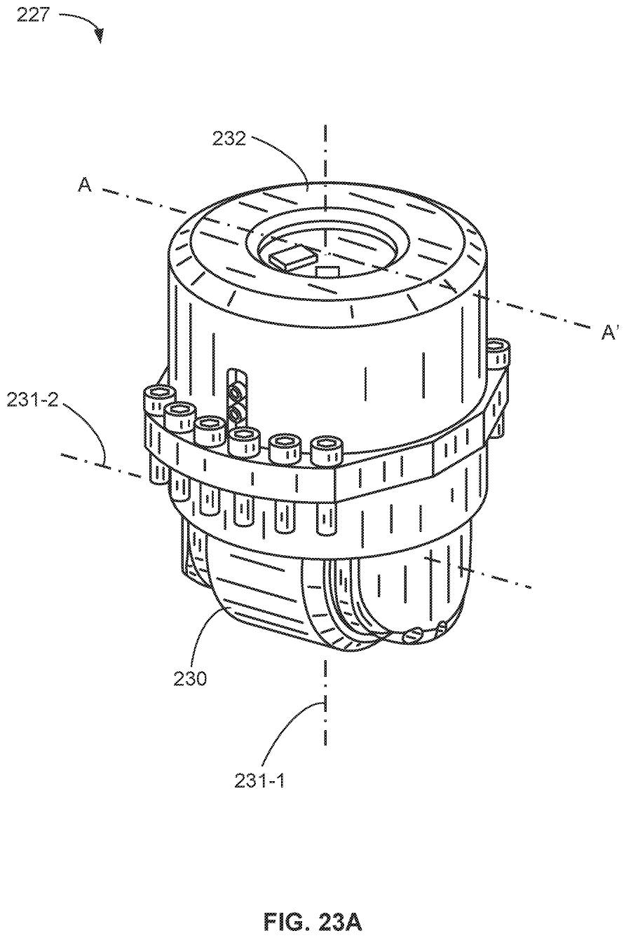

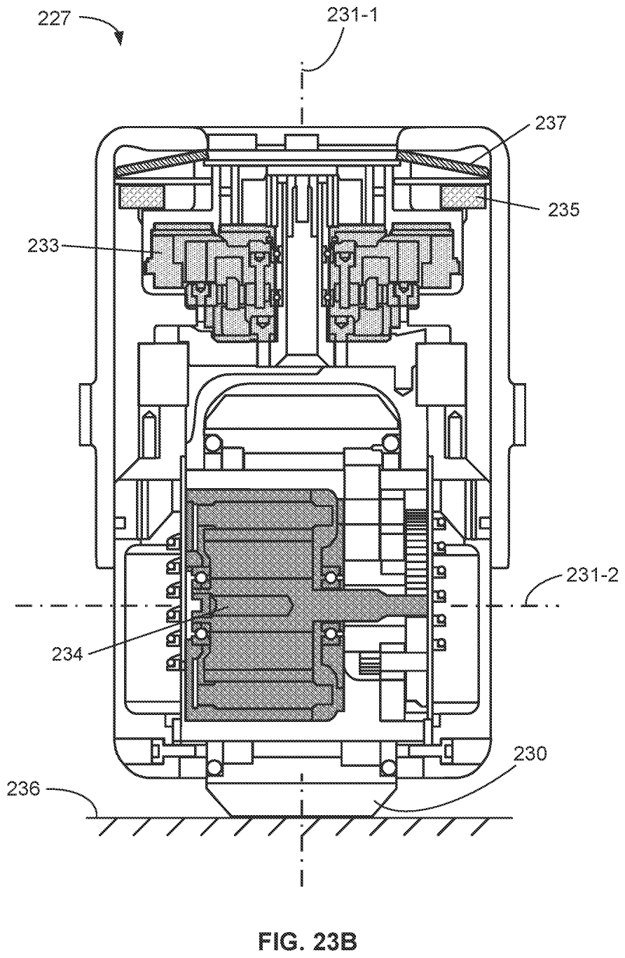

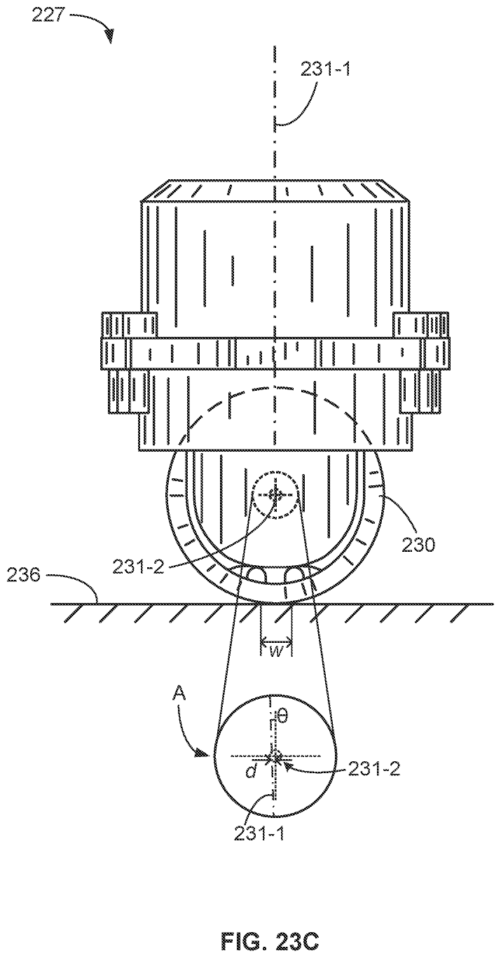

FIG. 23A illustrates a perspective view of a wheel assembly 227 of the mobile medical platform 200 of FIG. 21 in accordance with some embodiments. The wheel assembly 227 includes a wheel 230 that is configured to rotate around a rotational axis 231-1 and roll around a rolling axis 231-2. In some embodiments, mechanical components of the wheel assembly 227 are located inside a housing element 232 (e.g., an enclosure). A cross-sectional view of the wheel assembly 227 along AA' is shown in FIG. 23B, illustrating mechanical components of the wheel assembly 227 that power the wheel 230.

Referring to FIG. 23B, the wheel assembly 227 includes one or more motors configured to steer (e.g., rotate) and propel (e.g., roll) the wheel 230. The wheel assembly 227 shown in FIG. 23B includes a steering motor 233 that is configured to rotate or steer the wheel 230 around the rotational axis or roll axis 231-1, and a propulsion motor 234 that is configured to propel the wheel 230 by rolling the wheel 230 around the rolling axis 231-2. While the wheel assembly 227 shown in FIG. 23B includes two different (e.g., distinct and separate) motors for power steering and propulsion of the wheel 230, in some embodiments, power steering and propulsion of the wheel 230 may be provided by a single motor.

In some embodiments, the wheel 230 of the wheel assembly 227 is configured to rotate around the steering axis 231-1 in a clockwise and/or counter-clockwise direction. In some embodiments, the wheel 230 of the wheel assembly 227 is configured to rotate around the steering axis 231-1 by 360°. In some embodiments, the wheel 230 of the wheel assembly 227 is configured to rotate around the steering axis 231-1 by less than 360°, such as by 350°, 330°, 320°, 300°, 280°, 250°, 200°, 90°, 60°, 45°, 30°, or 15°. In some embodiments, the wheel 230 of the wheel assembly 227 is configured to rotate around the steering axis 231-1 by at least 180°.

In some embodiments, the propulsion motor 234 is configurable to propel the wheel 230 in both forward and backward directions (e.g., rolling the wheel 230 in clockwise and counterclockwise directions around the rolling axis 231-2). In some embodiments, the propulsion motor 234 is configurable to propel the wheel in one direction (e.g., only a clockwise or counterclockwise direction around the rolling axis 231-2).

In some embodiments, the wheel 230 is spring loaded, and the wheel assembly 227 includes a lower spring 235 (e.g., a suspension spring) that is positioned above the wheel 230 to continuously exert a downward force (either directly or indirectly) on the wheel 230. As a result, the lower spring 235 facilitates maintenance of contact between the wheel 230 and a ground or surface 236 while the wheel assembly 227 is in use (e.g., supporting the mobile medical platform 200). The lower spring 235 ensures that the wheel 230 remains in contact with the surface 236 regardless of a surface profile of the surface 236. For example, while the wheel assembly 227 is in use for transporting the mobile medical platform 200 or supporting the mobile medical platform 200 in a stationary position on an uneven surface (e.g., a floor with cracks, bumps, and/or holes), the lower spring 235 pushes the wheel 230 down in a direction toward the surface 236 such that the wheel 230 maintains contact with the surface 236 and provides stable support for the mobile medical platform 200. In some embodiments, the lower spring 235 is also positioned to exert an upward force (either directly or indirectly) on the first side 228 of the rigid base 221 and support the weight of the mobile medical platform 200.

In some embodiments, the wheel assembly 227 also includes an upper spring 237 (e.g., an energy absorbing spring or a shock absorbing spring) that is positioned above the wheel 230 to dampen relative movement between the wheel 230 and the rigid base 221 of the mobile medical platform 200, such as relative movement caused by the wheel 230 rolling over an uneven surface or a bump. The upper spring 237 is positioned between the wheel 230 and the rigid base 221. In configurations, in which wheel assembly 227 includes both the upper spring 237 and the lower spring 235, the upper spring 237 is located above the lower spring 235 such that the lower spring 235 is disposed between the upper spring 237 and the wheel 230. The upper spring 237 has a spring constant that is greater than a spring constant of the lower spring 235.

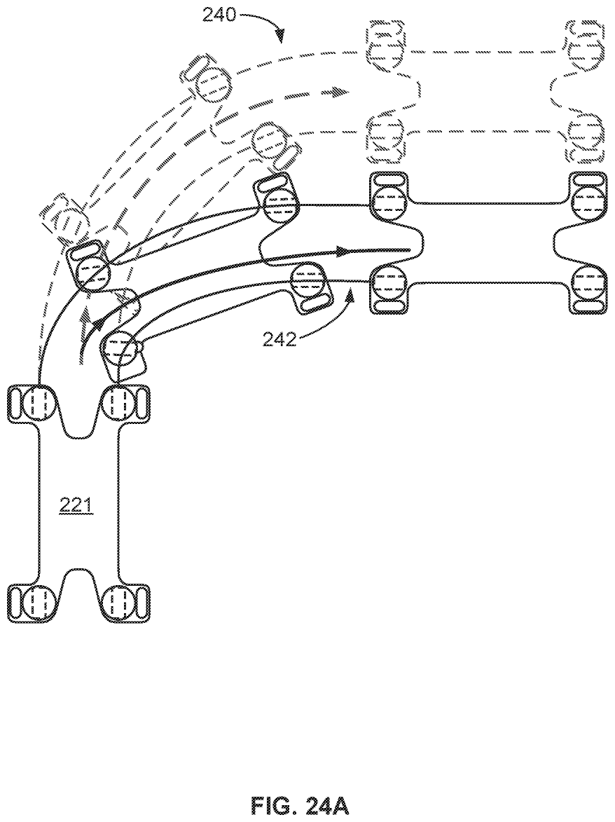

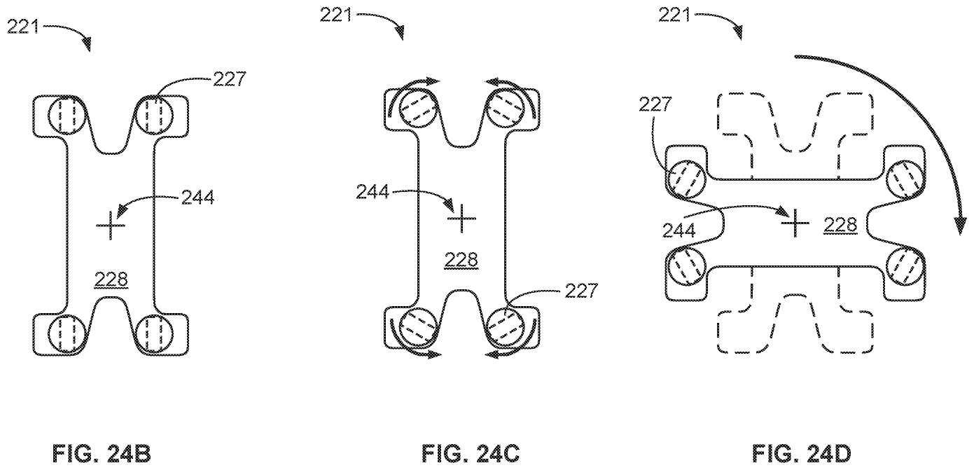

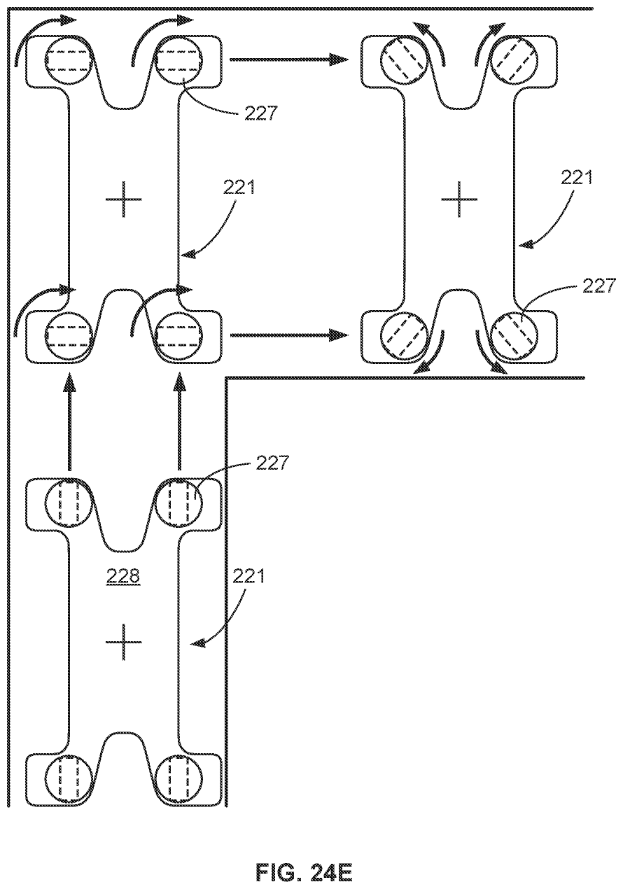

FIG. 23C illustrates a side view of the wheel assembly 227 of FIGS. 23A and 23B. The wheel 230 is positioned such that the rotational axis 231-1 of the wheel 230 and the rolling axis 231-2 of the wheel 230 has an offset distance d (called caster) and a caster angle θ that are negligible. Inset A, which is a zoomed-in view near the rotational axis 231-1 of the wheel 230, shows the offset distance d and the caster angle θ between the rotational axis 231-1 and the rolling axis 231-2 of the wheel 230. The offset distance d between the rotational axis 231-1 and the rolling axis 231-2 of the wheel 230 is considered negligible, substantially eliminated, or substantially zero when the offset distance d is 20% or less of the wheel's contact width w with the ground 236 (e.g., d<0.2w) or less than 6 millimeters. The contact width w of the wheel 230 is a distance between two furthest points on the wheel 230 that are in contact with the surface 236 under nominal or ideal conditions (e.g., when the surface 236 is flat). The offset distance d between the rotational axis 231-1 and the rolling axis 231-2 of the wheel 230 and the caster angle θ of the wheel 230 shown in FIG. 23C are negligible (e.g., substantially zero, substantially eliminated). By substantially eliminating the caster angle θ or offset distance d of the wheel 230, the wheel 230 is able to rotate around the rotational axis 231-1 with little or no swept volume and roll around the rolling axis 231-2 independently of the orientation of the wheel 230 with respect to the rotational axis 231-1. For example, the wheel 230 may be simultaneously rolled around the rolling axis 231-2 and rotated around rotational axis 231-1. The ability to propel the wheel 230 while simultaneously rotating the wheel 230 allows the wheel assembly to maneuver the mobile medical platform 200 in a number of ways that is not possible with conventional wheels that have a non-negligible caster angle. Examples of various movements that can be accomplished by a mobile medical platform 200 that is powered by one or more wheel assemblies 227 are described with respect to FIGS. 24A-24E.

FIG. 24A shows an example of two different turning paths that can be navigated by a mobile medical platform 200 that is powered by one or more wheel assemblies 227. A first turning path 240, shown with dashed lines, corresponds to a typical turning path of a mobile medical platform 200 that can be performed with or without the use of powered wheel assemblies 227. In contrast, a second turning path 242, shown with solid lines, corresponds to a turning path that has a smaller turning radius compared to the first turning path 240. Due to the ability of the wheels 230 of the powered wheel assemblies 227 to be simultaneously rotated and rolled, the second turning path 242 can be performed by a mobile medical platform 200 that includes one or more powered wheel assemblies 227, thereby allowing greater maneuverability of the mobile medical platform 200 around tight corners and small spaces.

In addition to being able to rotate and roll simultaneously, the wheel 230 of a powered wheel assembly 227 is also able to rotate without rolling. This allows the wheel 230 to be oriented in a desired direction prior to initiating rolling of the wheel 230 or movement of the mobile medical platform 200. For example, as shown in FIGS. 24B-24D, wheels 230 of the powered wheel assemblies 227 in mobile medical platform 200 are able to be rotated into a desired position prior to being rolled, thereby allowing the mobile medical platform 200 to be fully rotated or to pivot around a central position 244 without lateral translation of the mobile medical platform 200.

The ability to independently control rotation and propulsion of the wheel 230 of the powered wheel assembly 227 allows for precise movement of the mobile medical platform 200. For example, when navigating or positioning the mobile medical platform 200 in a tight space such as a small corridor or an elevator, it may be desirable to be able to orient the wheels 230 of wheel assemblies 227 while the mobile medical platform 200 is stationary. FIG. 24E illustrates an example of navigating a tight space (represented by thick lines) and precisely maneuvering the mobile medical platform 200 into a desired position. The wheels 230 of powered wheel assemblies 227 are able to be aligned in a same direction prior to moving the mobile medical platform 200 forward, and the wheels 230 can be re-oriented in a different direction prior to rolling the wheels 230 and moving the mobile medical platform 200 into the desired position. In addition to the path shown in FIG. 24E, the powered wheel assemblies 227 of the mobile medical platform 200 are able to transport the mobile medical platform 200 through the corner shown in FIG. 24E in many other ways, including turning and/or pivoting the mobile medical platform 200. The freedom to independently rotate and propel the wheels 230 of powered wheel assemblies 227 allows for a wide variety of movements depending on the desired final position and orientation of the mobile medical platform 200. Thus, a user may easily maneuver the mobile medical platform 200 in a multitude of physical environments with precision and ease.

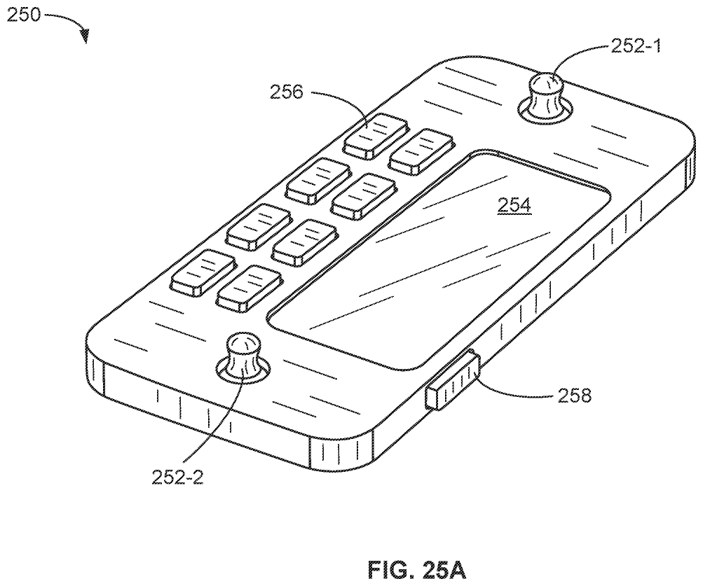

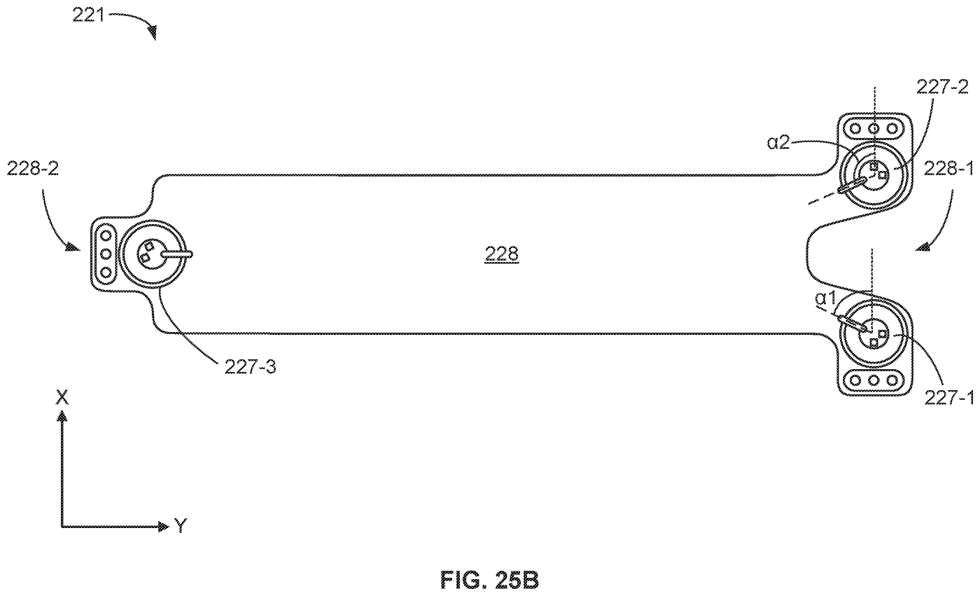

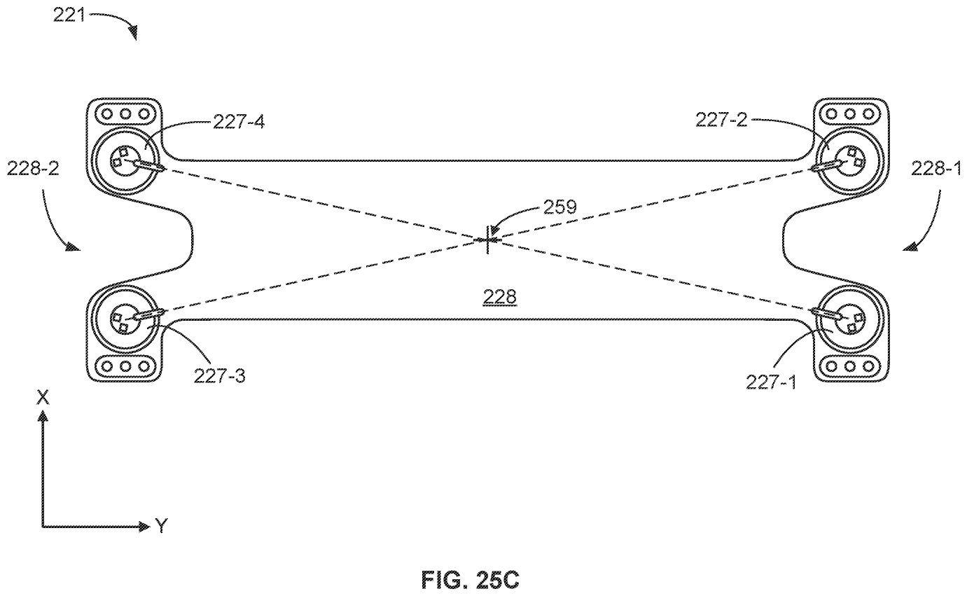

In some embodiments, a user or operator may control movement of the mobile medical platform 200 via one or more input devices such as a handheld pendant or controller. FIG. 25A illustrates an example of an input device 250 for controlling movement of the mobile medical platform 200. The input device 250 may communicate with the mobile medical platform 200 via a wireless connection, such as Bluetooth or over a wireless network, or via one or more wired electrical connections. Thus, the input device 250 may be implemented in different ways—the input device 250 may be a handheld pendant, a controller, a joystick controller, or even a device with a touch screen surface, such as a tablet or a smart phone. For example, the input device 250 may be located on or mounted onto the mobile medical platform 200 and the input device 250 be electrically connected to each wheel assembly 227. In another example, the input device may be a smart phone or a tablet that is in communication with the mobile medical platform 200 via a wireless network or a Bluetooth connection. The smart phone or tablet may include an application that is configured to allow a user to control movement of the mobile medical platform 200 via user inputs at a touch screen or affordance of the smart phone or tablet. In some embodiments, the input device 250 is able to communicate with the mobile medical platform 200 within a predefined range of operation (e.g., the mobile medical platform 200 and the input device are within 5 feet, 10 feet, 20 feet, 50 feet of one another). Depending on the implementation of the input device, an operator of the mobile medical platform 200 may push the mobile medical platform 200 or control movement of the mobile medical platform 200 while walking alongside the mobile medical platform 200 while the mobile medical platform 200 is, for example, used to transport a patient.

The input device 250 includes a steering affordance 252-1 configured to control an orientation (e.g., direction, heading) of the mobile medical platform 200, and a driving affordance 252-2 configured to control a motion of the mobile medical platform 200. For example, by providing an input via the steering affordance 252-1, a user may cause the mobile medical platform 200 to rotate or pivot clockwise or counterclockwise with negligible lateral movement (e.g., without lateral translation of the mobile medical platform 200). In addition, by providing an input via the driving affordance 252-2, a user may cause the mobile medical platform 200 move laterally in any direction (left, right, forwards, backwards, diagonally) without rotating or changing an orientation of the mobile medical platform 200. Operations of each wheel assembly 227 of the mobile medical platform 200 are automatically controlled and coordinated to achieve a movement or action as requested by a user via the input device 250. Although the steering affordance 252-1 and the driving affordance 252-2 are shown as physical joysticks in FIG. 25A, the steering affordance 252-1 and the driving affordance 252-2 may be implemented via a touch screen or a touch pad or replaced by directional affordances (e.g., affordances corresponding to left, right, forward, and backward directions).

In some embodiments, the input device 250 includes a display 254. The display 254 may present a representation of the mobile medical platform 200 and/or may show additional information regarding the mobile medical platform 200 such as any warning or error messages, for example, a low battery warning. The input device 250 optionally includes one or more additional affordances 256. An affordance of the one or more additional affordances 256 may correspond to other functions of the mobile medical platform 200, such as changing a height of a table top 225 relative to the rigid base 221 of the mobile medical platform 200 or a preset setting of the mobile medical platform 200.

In some embodiments, the input device 250 includes a motion affordance 258. The motion affordance 258 may be associated with preset criteria for mobilizing or braking the mobile medical platform 200. For example, preset criteria may require detection of user input at the motion affordance 258 (e.g., the motion affordance 258 is activated, initiated, pressed, depressed) in order to initiate movement of the mobile medical platform 200 or in order to initiate propulsion or orientation of the wheels 230 of the wheel assemblies 227. In another example, the preset criteria may require that user input at the motion affordance 258 is maintained (e.g., constantly depressed, activated, pressed) while the mobile medical platform 200 and/or the wheels 230 of the wheel assemblies 227 are in motion. In a third example, the preset criteria may require detection of user input at any of the steering affordance 252-1 and the driving affordance 252-2. In a fourth example, the preset criteria may require that the mobile medical platform 200 be operated below a predetermined speed (e.g., movement of the mobile medical platform 200 does not exceed the predetermined speed). In yet another example, the preset criteria may require that two or more of the aforementioned conditions be satisfied.

In some embodiments, in response to a determination that preset criteria are met (e.g., a user input continues to be detected at the motion affordance 258, user input is received at the steering affordance 252-1 and/or the driving affordance 252-2), wheels 230 of the wheel assemblies 227 are automatically oriented (e.g., in same direction) based on a previous user input so that the mobile medical platform 221 continues to move.

In some embodiments, in response to a determination that preset criteria are not met (e.g., user input is not detected at the motion affordance 258, ceasing user input at the motion affordance 258, user input is not detected at any of the steering affordance 252-1 and the driving affordance 252-2, or mobile medical platform 200 is traveling at a speed that exceeds the predetermined speed), the mobile medical platform may automatically engage in an immobilization setting. For example, the mobile medical platform 200 automatically orients wheels 230 of the wheel assemblies 227 in a preset braking configuration, thereby immobilizing the mobile medical platform 200 and preventing unintentional movement of the mobile medical platform 200. In some embodiments, the immobilization setting may correspond to a change in a motor speed or motor direction of the propulsion motor 224, resulting in a slowing down or braking of the wheel 230.

In some embodiments, the mobile medical platform 200 includes one or more brake pads that are configured to make contact with the wheel 230 in order to slow or stop a rolling motion of the wheel 230. In some embodiments, the mobile medical platform 200 includes a deployable lever-based breaking mechanism that when deployed, is in contact with a floor (such as surface 236) and lifts the rigid base 221 so one or more wheels 230 of the wheel assemblies 227 are not in contact with the floor. FIGS. 25B and 25C illustrate examples of a preset braking configuration of the mobile medical platform of FIGS. 22B and 22A, respectively.

As shown in FIG. 25B, when the mobile medical platform 200 includes two or more wheel assemblies (in this example, three wheel assemblies, 227-1 through 227-3), the preset braking configuration corresponds to wheels 230 of at least two different wheel assemblies 227 being oriented in different directions (represented by dashed lines) so that the respective wheels 230 are not parallel to one another. For example, wheel 230 of wheel assembly 227-1 is oriented in a first direction or at a first angle α1 relative to a reference axis and the wheel 230 of wheel assembly 227-2 is oriented in a second direction or at a second angle α2, that is different from the first angle, relative to the reference axis. In some embodiments, the first angle and the second angle may differ by a range of 10 degrees to 170 degrees.

In a configuration, in which the mobile medical platform 200 includes four wheel assemblies, the preset braking configuration corresponds to wheels 230 of adjacent wheel assemblies 227 (e.g., wheel assemblies 227 that are on a same end, a front end 228-1 or a back end 228-2, of the mobile medical platform 200) being oriented in different directions. In some embodiments, the wheels 230 of the four wheel assemblies are directed to a common point, such as a centroid 259 of the at least four wheel assemblies, while in the preset braking configuration. For example, as shown in FIG. 25C, the wheels 230 of the four wheel assemblies 227 are oriented to form an “X” shape when in the preset braking configuration. In some embodiments, the wheels of the wheel assemblies 227-1 and 227-2 are directed to a first common point and the wheel of the wheel assemblies 227-3 and 227-4 are directed to a second common point that is distinct from the first common point.

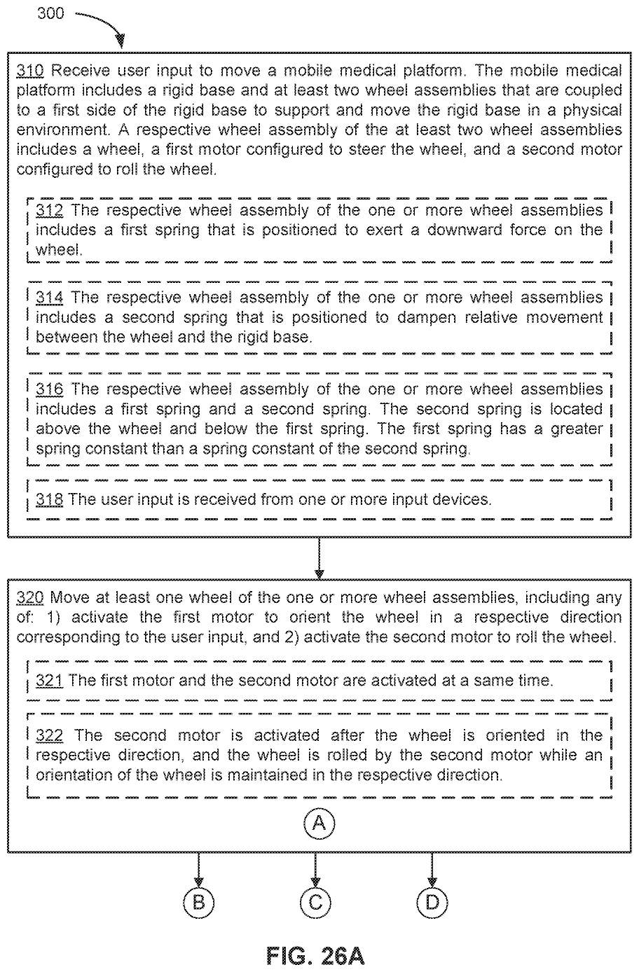

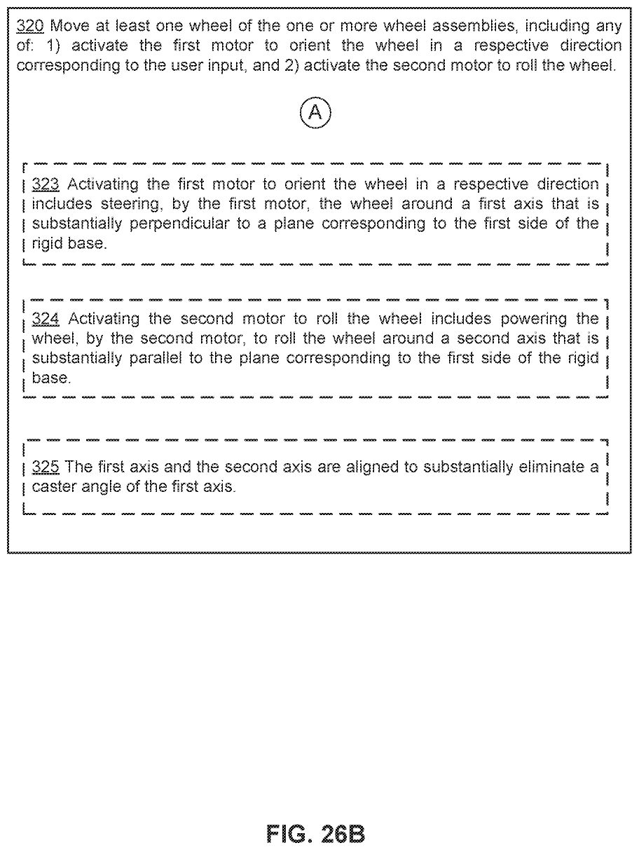

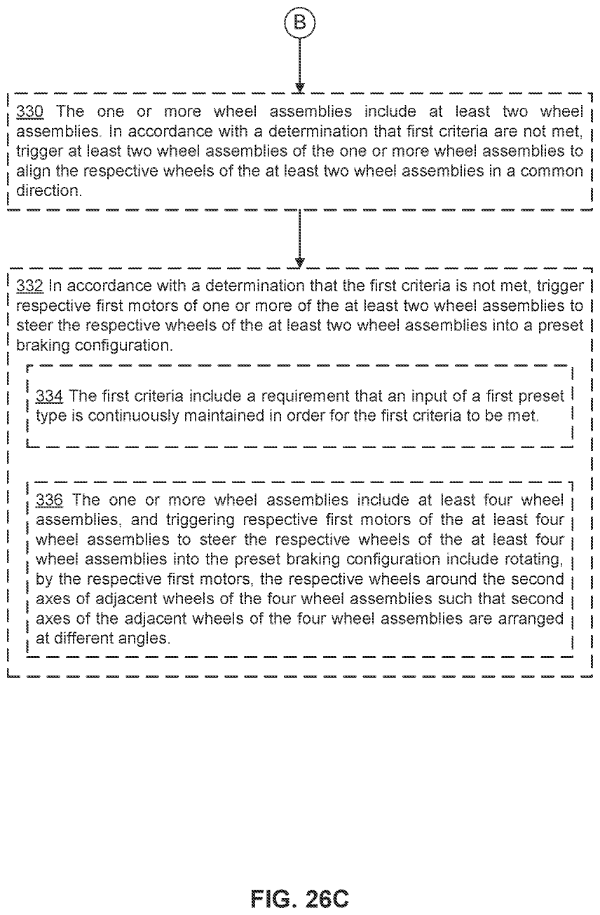

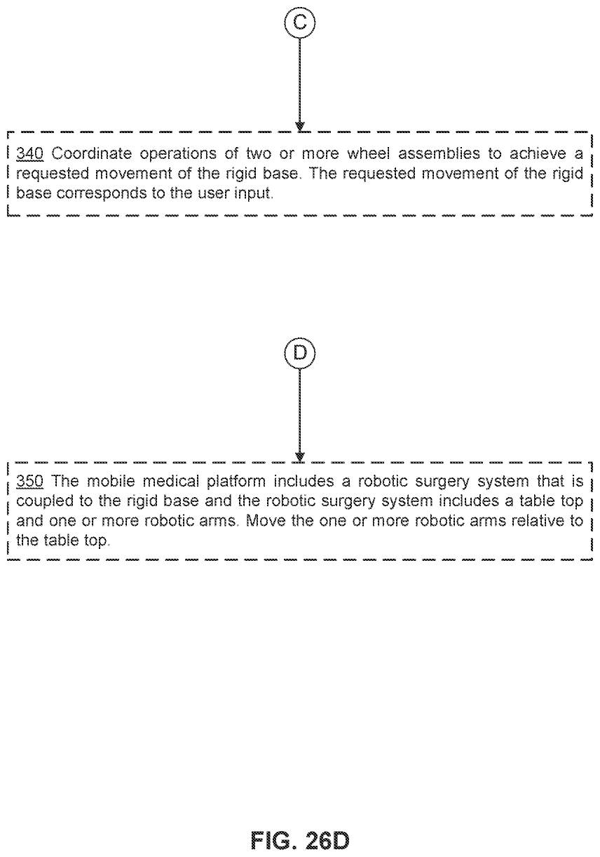

FIGS. 26A-26D show a flowchart illustrating a method 300 performed by a mobile medical platform (e.g., mobile medical platform 200) in accordance with some embodiments.

The mobile medical platform 200 (e.g., a surgical bed, surgical table, robotic surgical table) includes a rigid base 221 (e.g., a table base for a surgical bed, a rigid load-bearing housing, a chassis) and one or more wheel assemblies 227 that are coupled (e.g., rigidly coupled) to a first side 228 of the rigid base 221 to support and move the rigid base 221 in a physical environment. A respective wheel assembly 227 of the one or more wheel assemblies includes a wheel 230, a first motor 223 (e.g., a steering motor) configured to steer the wheel, and a second motor 234 (e.g., a propulsion motor) configured to roll the wheel 230.

In some embodiments, the mobile medical platform 200 is a surgical bed that includes a table top 225 and a rigid base 221 supporting the table top 225. In some embodiments, the mobile medical platform 200 further includes medical equipment (e.g., robotic arms 205 in docked or undocked positions, monitoring equipment attached to the patient that is being transported by the mobile medical platform 200) that are supported by the rigid base 221. In some embodiments, the mobile medical platform 200 supports a patient during movement of the mobile medical platform 200, while an operator of the mobile medical platform 200 pushes the mobile medical platform 200 or controls movement of the mobile medical platform 200 while walking alongside the mobile medical platform 200.

The method 300 includes (310) receiving user input to move the mobile medical platform, and (320) moving at least one wheel 230 of the one or more wheel assemblies 227, including any of: A) activating the first motor 223 to orient (e.g., rotate or steer) the wheel 230 in a respective direction corresponding to the user input, or B) activating the second motor 234 to roll (e.g., propel) the wheel 230. In some embodiments, at least one wheel 230 of the one or more wheel assemblies 227 is moved in accordance with one or more inputs, such as a user input (e.g., a user requested movement of the mobile medical platform 200), sensor information, bed positon information, and/or bed motion information.

In some embodiments, the respective wheel assembly 227 of the one or more wheel assemblies includes (312) a first spring 235 (e.g., lower spring 235) positioned to exert a downward force (e.g., directly or indirectly) on the wheel 230. As a result, the first spring 235 facilitates the wheel 230 to maintain contact with a floor (e.g. surface 236). The first spring 235 is positioned to exert an upward force (e.g., directly or indirectly) on the first side 228 of the rigid base 221 of the mobile medical platform 200 and supports the weight of the mobile medical platform 220.

In some embodiments, the respective wheel assembly 227 of the one or more wheel assemblies includes (314) a second spring 237 (e.g., upper spring 237) that is positioned to dampen relative movement between the wheel 230 and the rigid base 221 (e.g., relative movement caused by the wheel 230 rolling over bumps and/or holes in an uneven surface).

In some embodiments, the respective wheel assembly 227 of the one or more wheel assemblies includes (316) a first spring 235 (e.g., an upper spring or energy absorbing spring 235) and a second spring 237 (e.g., a lower spring or a suspension spring 237). The first spring 235 is located below the rigid base 221 and above the second spring 237. The second spring 237 is located above the wheel 230 and below the first spring 235. The first spring 235 has a greater spring constant than a spring constant of the second spring 237 (e.g., the first spring 235 is stiffer than the second sprint 237).

In some embodiments, the user input is received (318) from one or more input devices (e.g., input device 250). In some embodiments, the one or more input devices are in communication (e.g., wired or wireless communication) with the mobile medical platform 200.

In some embodiments, the first motor 233 and the second motor 234 are activated (321) at a same time so that the wheel 230 is simultaneously rotated and rolled (e.g., simultaneously steered and propelled). Such simultaneous steering and rolling operations facilitate smooth transportation of the mobile medical platform.

In some embodiments, the second motor 234 is activated (322) after the wheel 230 is oriented in the respective direction (e.g., by the first motor 233), and the wheel 230 is rolled by the second motor 234 while an orientation of the wheel 230 is maintained in the respective direction (e.g., by the first motor 233). For example, a wheel 230 may be steered by a first motor 233 in a desired direction prior to being propelled forward by the second motor 234 in the desired direction. Such sequential steering and rolling facilitates accurate positioning of the mobile medical platform.

In some embodiments, activating the first motor 233 to orient the wheel 230 in a respective direction includes (323) steering, by the first motor 233, the wheel 230 around a first axis 231-1 (e.g., a rotational axis or a steering axis 231-1) that is substantially perpendicular to a plane corresponding to the first side 228 of the rigid base 221.

In some embodiments, activating the second motor 234 to roll the wheel 230 includes (324) powering the wheel 230, by the second motor 234, to roll the wheel 230 around a second axis 231-2 (e.g., a rolling axis 231-2) that is substantially parallel to the plane corresponding to the first side 228 of the rigid base 221.

In some embodiments, (325) the first axis 231-1 and the second axis 231-2 are aligned to substantially eliminate a caster angle θ of the first axis 231-1 (also referred to herein as a caster angle θ of the wheel 230) such that an offset distance d between the first axis 231-1 and the second axis 231-2 is substantially zero. A caster angle θ of the first axis 231-1 (or the wheel 230) is considered to be substantially eliminated when an offset distance d between the first axis 231-1 and the second axis 231-2 is 20% or less of the wheel's contact width w with the ground 236 (e.g., d<0.2w) or less than 6 millimeters. For example, first axis 231-1 intersects with the second axis 231-2 such that the offset distance d is zero. Illustration and discussion of the caster angle θ of the first axis 231-1 (or the wheel 230), the offset distance d, and the wheel's contact width w are provided with respect to FIG. 23C. Such elimination or reduction of the caster angle facilitates steering of the wheel 230 by the first motor.

In some embodiments, the one or more wheel assemblies 227 include at least two wheel assemblies (e.g., the mobile medical platform includes two wheel assemblies on the front end 280-1 of the rigid base 221, three wheel assemblies as shown in FIG. 22B, four wheel assemblies on the four quadrants of the rigid base 221 as shown in FIG. 22A, or five or more wheel assemblies), and the method 300 includes, in accordance with a determination that the first criteria are met (e.g., preset automatic-braking criteria are not met), (330) triggering respective first motors 233 of one or more of the at least two wheel assemblies 227 to steer the respective wheels 230 of the at least two wheel assemblies 227 in a common direction (e.g., with their rolling axes parallel to one another) in accordance with a determination that first criteria are met (e.g., motion affordance 258 or a dead-man switch is depressed, and/or a driving input and/or steering input is received from a user at a user device 250).

In some embodiments, the method 300 includes, in accordance with a determination that first criteria are not met (e.g., preset automatic-braking criteria are met, a motion affordance 258 or dead-man switch is released, the rigid base 221 is moving faster than a preset or threshold speed), triggering (332) respective first motors 233 of one or more of the at least two wheel assemblies (e.g., wheel assemblies 227-1 and 227-2) to steer (e.g., align) respective wheels 230 of the at least two wheel assemblies 227 in a preset braking configuration. For example, the second axes 231-2 (e.g., rolling axes 231-2) of wheels 230 on a same side of the rigid base 221 (e.g., wheels 230 on a front end 280-1 of the rigid base 221, wheels 230 on a back end 280-2 of the rigid base 221, wheels 230 on a left side of the rigid base 221, wheels 230 on a right side of the rigid base 221) are perpendicular to the second axis of at least one other wheel 230. An example of a preset braking configuration of wheels in a mobile medical platform having at least four wheel assemblies is provided with respect to FIG. 25B.

In some embodiments, the first criteria include (334) a requirement that an input of a first preset type is continuously maintained in order for the first criteria to be met. For example, the first criteria may require that a motion affordance 258 on an input device 250 be continuously pressed (e.g., depressed, activated) or pressed at all times during movement of the mobile patient platform 200 in order to permit movement of the mobile medical platform 200.

In some embodiments, the one or more wheel assemblies 227 includes at least four wheel assemblies (e.g., the mobile medical platform includes four or more wheel assemblies). Triggering respective first motors 233 of the at least four wheel assemblies 227 to steer the respective wheels 230 of the at least four wheel assemblies 227 into the preset braking configuration include rotating (336), by the respective first motors 233, the respective wheels 230 around the second axes 231-2 of adjacent wheels 230 of the four wheel assemblies 227 such that second axes 231-2 of the adjacent wheels of the four wheel assemblies 227 are arranged at different angles. For example, the rolling axes 231-2 of the wheels 230 on a same side of the rigid base 221 base (e.g., wheels 230 on a front end 280-1 of the rigid base 221, wheels 230 on a back end 280-2 of the rigid base 221, wheels 230 on a left side of the rigid base 221, wheels 230 on a right side of the rigid base 221) are oriented at an angle (e.g., 90 degrees, 60 degrees, etc.) relative to each other and are not parallel to each other. In another example, the wheels 230 can be oriented to form an “X” shape on the bottom 228 of the rigid base 221 as shown in FIG. 25C when in the wheels 230 are in the preset braking configuration.

In some embodiments, the mobile medical platform includes one or more deployable levers, which, when deployed, are in contact with a floor (e.g., surface 236) and lift the rigid base 221 so that wheels 230 of the one or more wheel assemblies 227 cease to be in contact with the floor.

In some embodiments, the method 300 further includes (340) coordinating operations of two or more wheel assemblies 227 to achieve a requested movement of the rigid base 221. The requested movement of the rigid base 221 corresponds to the user input. For example, directions of the wheels 230 may be coordinated, as shown in FIG. 24A, to steer the mobile medical platform 220 during forward movement. In another example, as shown in FIGS. 24B-24D, the wheels 230 of the two or more wheel assemblies 227 can be coordinated to provide rotational movement (e.g., pivot) with zero or negligible translated of the rigid base 221 relative to the physical environment.

In some embodiments, the mobile medical platform 200 includes a robotic surgery system (e.g., surgical robotics system 100) that is coupled to the rigid base 221 and the robotic surgery system includes a table top 225 (such as a surgical table top) and one or more robotic arms 205. The method 300 may also include (350) moving the one or more robotic arms 205 relative to the table top 225.

In some embodiments, the method includes receiving one or more control parameters (e.g., direction, displacement, translation, preset instruction) corresponding to user input (e.g., press of a button, swipe input, movement, gesture) from one or more input devices (e.g., a joy stick, touch-screen device, control device, etc.) that are in communication with the mobile medical platform 200 (e.g., in communication with one or more processors of the mobile medical platform 200), and controlling respective first motors 233 and respective second motors 234 of the one of more wheel assemblies 227 to move respective wheels 230 in accordance with the one or more control parameters.

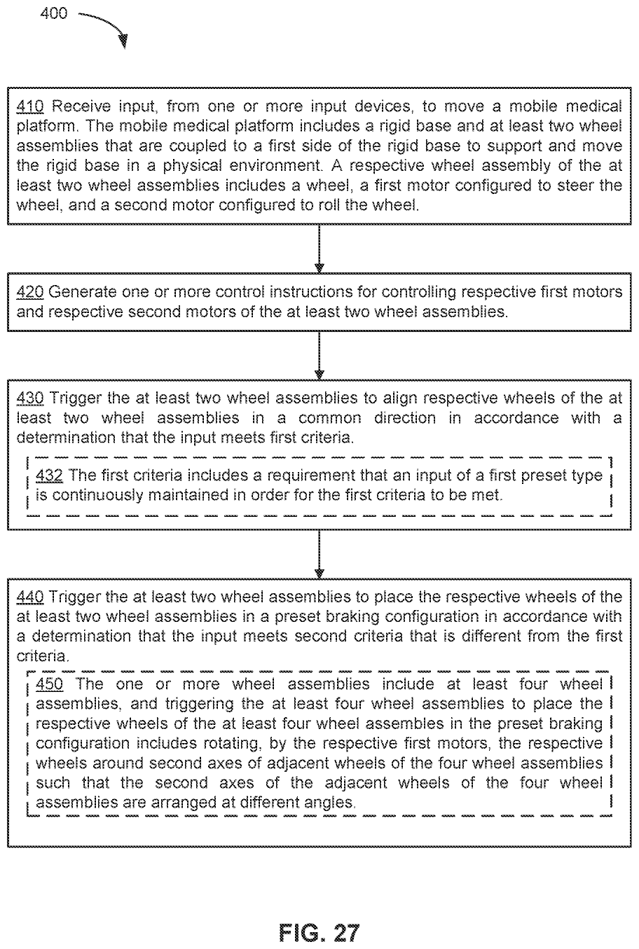

FIG. 27 is a flowchart illustrating another method 400 for utilizing a mobile medical platform (e.g., mobile medical platform 200) in accordance with some embodiments. The mobile medical platform 200 includes a rigid base 221 and at least two wheel assemblies 227 that are coupled to a first side 228 of the rigid base 221 to support and move the rigid base 221 in a physical environment. A respective wheel assembly 227 of the at least two wheel assemblies includes a wheel 230, a first motor 233 configured to steer the wheel 230, and a second motor 234 configured to roll the wheel.

The method 400 includes (410) receiving input (e.g., sensor information, bed position/motion information, user input) from one or more input devices (e.g., input device 250). In some embodiments, the input corresponds to a request to move a mobile medical platform 200.

The method 400 also includes (420) generating one or more control instructions for controlling respective first motors 233 and respective second motors 234 of the at least two wheel assemblies 227. Generating the one or more control instructions includes (430) triggering the at least two wheel assemblies 227 to align respective wheels 230 of the at least two wheel assemblies 227 in a common direction in accordance with a determination that the input meets first criteria.

In some embodiments, the first criteria include a requirement that an input of a first preset type is continuously maintained in order for the first criteria to be met, such as depressing a motion affordance 258 while providing one or more user inputs regarding movement of the mobile medical platform 200.

Generating the one or more control instructions includes (440) triggering the at least two wheel assemblies 227 to place the respective wheels 230 of the at least two wheel assemblies 227 in a preset braking configuration in accordance with a determination that the input meets second criteria that is different from the first criteria. For example, when the motion affordance 258 is not depressed, respective wheels 230 of the at least two wheel assemblies 227 are oriented in a preset braking configuration that immobilizes the mobile medical platform 200.

In some embodiments, the one or more wheel assemblies 227 include at least four wheel assemblies (e.g., the mobile medical platform includes at least four wheel assemblies). Triggering the at least four wheel assemblies 227 to place the respective wheels 230 of the at least four wheel assemblies 227 in the preset braking configuration includes rotating (450), by the respective first motors 233, the respective wheels 230 around second axes 231-2 of adjacent wheels of the four wheel assemblies 227 such that the second axes 231-2 of the adjacent wheels of the four wheel assemblies 227 are arranged at different angles.

In some embodiments, the respective wheels 230 of the at least four wheel assemblies 227 are directed to a common point while in the braking configuration. In some embodiments, the common point is a centroid 259 of the at least four wheel assemblies 227. For example, respective wheels 230 of the four wheel assemblies 227 may be arranged into an “X” formation, as illustrated in FIG. 25C.

As described herein, a wheel assembly of a mobile medical platform 200 may have a negligible caster (and a negligible caster angle). The reduction or elimination of the caster allows transportation of the mobile medical platform with little or no swept volume, which, in turn, improves the positioning accuracy in transporting the mobile medical platform. It also facilitates independent selection of the steering direction for each wheel, which simplifies the control mechanism.

In accordance with some embodiments, a mobile medical platform 200 includes a rigid base 221 and one or more wheel assemblies 227 that are coupled to a first side 228 of the rigid base 221 and support the rigid base 221. A respective wheel assembly 227 of the one or more wheel assemblies includes a wheel 230 that is configured to respectively rotate around a first axis 231-1 (e.g., rotational or steering axis 231-1) and a second axis 231-2 (e.g., rolling axis 231-2) that is different from the first axis 231-1, and a first motor positioned for rotating the wheel 230 around a respective one of the first axis 231-1 and the second axis 231-2. The first axis 231-1 is aligned with the second axis 231-2, resulting in a negligible caster angle θ of the wheel 230. In some embodiments, the respective wheel assembly 227 includes a second motor that is distinct from the first motor. In some embodiments, the respective wheel assembly 227 does not include the second motor that is distinct from the first motor.

In some embodiments, the mobile medical platform 200 further includes one or more processors and memory storing instructions, which, when executed by the one or more processors, cause the first motor 233 to move the wheel 230 in accordance with one or more inputs.

In some embodiments, the respective wheel 230 assembly further includes a second motor 234. The stored instructions, when executed by the one or more processors, cause the one or more processors to receive one or more control parameters corresponding to user from one or more input devices (e.g., input device 260) that are in communication with the one or more processors, and control respective first motors 233 and respective second motors 234 of the one or more wheel assemblies 227 to move respective wheels 230 of the one or more wheel assemblies 227 in accordance the one or more control parameters.

As described herein, a wheel assembly of a mobile medical platform may include a combination of two springs. The combination of the two springs facilitates that a respective wheel remains in contact with a floor while dampening shocks or vibrations caused by a non-flat floor surface.

In accordance with some embodiments, a mobile medical platform 200 includes a rigid base 221 and one or more wheel assemblies 227 that are coupled to a first side 228 of the rigid base 221 and support the rigid base 221 during movement of the mobile medical platform 200. A respective wheel assembly 227 of the one or more wheel assemblies includes a wheel 230, a first motor 233 positioned for rotating the wheel, a first spring 235 positioned to exert a downward force on the wheel 230, and a second spring 237 positioned to dampen relative movement between the wheel 230 and the rigid base 221. In some embodiments, the respective wheel assembly 227 includes a second motor 234 for rolling the wheel. In some embodiments, the respective wheel assembly 227 does not include the second motor 234 for rolling the wheel.

In some embodiments, the first motor 223 is positioned for rotating the wheel 230 around a first axis 231-1 (e.g., rotational or steering axis 231-1) that is substantially perpendicular to a plane corresponding to the first side 228 of the rigid base 221. The respective wheel assembly of the one or more wheel assemblies also includes a second motor 235 positioned for rolling the wheel 230 around a second axis 231-2 (e.g., rolling axis 231-2) that is substantially parallel to the plane corresponding to the first side 228 of the rigid base 221.

In some embodiments, the mobile medical platform 200 includes one or more processors and memory storing instructions, which, when executed by the one or more processors, cause at least one of the first motor 233 or the second motor 235 to move the wheel 230 in accordance with one or more inputs.

In accordance with some embodiments, a mobile medical platform 200 includes a rigid base 221 and at least four wheel assemblies 227 that are coupled to the rigid base 221 and support the rigid base 221. A respective wheel assembly 227 of the at least four wheel assemblies includes a respective wheel 230 and a respective first motor 223 positioned for steering the respective wheel. The mobile medical platform 200 also includes one or more processors and memory storing instructions, which, when executed by the one or more processors, cause steering of the respective wheels 230 of the at least four wheel assemblies 227 such that the respective wheels 230 of the at least four wheel assemblies 227 are aligned in a common direction at a first time, and the respective wheels 230 of the at least four wheel assemblies 227 are arranged in a braking configuration, at a second time distinct from the first time, so that the rigid base 221 is immobilized. In some embodiments, the respective wheel assembly 227 includes a second motor for rolling the respective wheel. In some embodiments, the respective wheel assembly 227 does not include the second motor for rolling the respective wheel.

In some embodiments, the respective wheels 230 of the at least four wheel assemblies 227 are directed to a common point while in the braking configuration.

In some embodiments, the common point is a centroid 259 of the at least four wheel assemblies 227.

Embodiments of the disclosure relate to systems and techniques for providing power assisted mobility for a mobile medical platform, such as a hospital bed or a surgical table.

### 3. Implementing Systems and Terminology.

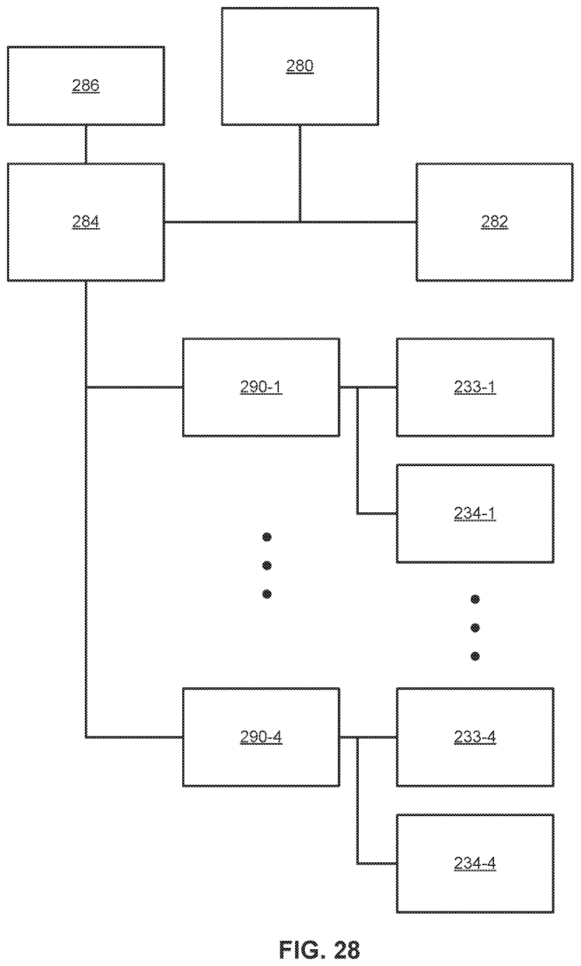

FIG. 28 is a schematic diagram illustrating electronic components of the mobile medical platform 200 in accordance with some embodiments.

The mobile medical platform 200 includes one or more processors 280, which are in communication with a computer readable storage medium 282 (e.g., computer memory devices, such as random-access memory, read-only memory, static random-access memory, and non-volatile memory, and other storage devices, such as a hard drive, an optical disk, a magnetic tape recording, or any combination thereof) storing instructions for performing any methods described herein (e.g., operations described with respect to FIGS. 26A-26D and 27). The one or more processors 280 are also in communication with an input/output controller 284 (via a system bus or any electrical circuit). The input/output controller 284 receives instructions and/or data from an input device (e.g., a user input device 286 that corresponds to input device 250) and relays the received instructions and/or data to the one or more processors 280 (e.g., with or without any translation, conversion, and/or data processing). The input/output controller 284 also receives instructions and/or data from the one or more processors 280 and relays the instructions and/or data to one or more actuators, such as first motors 233-1 through 233-4 and second motors 234-1 through 234-4. In some embodiments, the input/output controller 284 is coupled to one or more actuator controllers 290-1 through 290-4 and provides instructions and/or data to at least a subset of the one or more actuator controllers 290-1 through 290-4, which, in turn, provide control signals to selected actuators. In some embodiments, the one or more actuator controller 290-1 through 290-4 are integrated with the input/output controller 284 and the input/output controller 284 provides control signals directly to the one or more actuators (without a separate actuator controller). Although FIG. 28 shows that there are separate actuator controllers 290-1 through 290-4 (e.g., one actuator controller for each wheel assembly), in some embodiments, fewer actuator controllers may be used (e.g., one actuator controller for the entire mobile medical platform, or one actuator controller for a pair of wheel assemblies, etc.), additional actuator controllers may be used (e.g., one actuator controller for each actuator, such as a first motor or a second motor), or any combination thereof.

Implementations disclosed herein provide systems, methods and apparatus for medical platforms with power-assisted mobility.

It should be noted that the terms “couple,” “coupling,” “coupled” or other variations of the word couple as used herein may indicate either an indirect connection or a direct connection. For example, if a first component is “coupled” to a second component, the first component may be either indirectly connected to the second component via another component or directly connected to the second component.

The functions for power-assisted mobilization of a mobile medical platform described herein may be stored as one or more instructions on a processor-readable or computer-readable medium. The term “computer-readable medium” refers to any available medium that can be accessed by a computer or processor. By way of example, and not limitation, such a medium may comprise random access memory (RAM), read-only memory (ROM), electrically erasable programmable read-only memory (EEPROM), flash memory, compact disc read-only memory (CD-ROM) or other optical disk storage, magnetic disk storage or other magnetic storage devices, or any other medium that can be used to store desired program code in the form of instructions or data structures and that can be accessed by a computer. It should be noted that a computer-readable medium may be tangible and non-transitory. As used herein, the term “code” may refer to software, instructions, code or data that is/are executable by a computing device or processor.

The methods disclosed herein comprise one or more steps or actions for achieving the described method. The method steps and/or actions may be interchanged with one another without departing from the scope of the claims. In other words, unless a specific order of steps or actions is required for proper operation of the method that is being described, the order and/or use of specific steps and/or actions may be modified without departing from the scope of the claims.

As used herein, the term “plurality” denotes two or more. For example, a plurality of components indicates two or more components. The term “determining” encompasses a wide variety of actions and, therefore, “determining” can include calculating, computing, processing, deriving, investigating, looking up (e.g., looking up in a table, a database or another data structure), ascertaining and the like. Also, “determining” can include receiving (e.g., receiving information), accessing (e.g., accessing data in a memory) and the like. Also, “determining” can include resolving, selecting, choosing, establishing and the like.

The phrase “based on” does not mean “based only on,” unless expressly specified otherwise. In other words, the phrase “based on” describes both “based only on” and “based at least on.”

The previous description of the disclosed implementations is provided to enable any person skilled in the art to make or use the present invention. Various modifications to these implementations will be readily apparent to those skilled in the art, and the generic principles defined herein may be applied to other implementations without departing from the scope of the invention. For example, it will be appreciated that one of ordinary skill in the art will be able to employ a number corresponding alternative and equivalent structural details, such as equivalent ways of fastening, mounting, coupling, or engaging tool components, equivalent mechanisms for producing particular actuation motions, and equivalent mechanisms for delivering electrical energy. Thus, the present invention is not intended to be limited to the implementations shown herein but is to be accorded the widest scope consistent with the principles and novel features disclosed herein.

Some embodiments or implementations are described with respect to the following clauses:

Clause 1. A mobile medical platform, comprising: a rigid base; and one or more wheel assemblies that are coupled to a first side of the rigid base to support and move the rigid base in a physical environment, wherein a respective wheel assembly of the one or more wheel assemblies includes: a wheel, a first motor that is configured to steer the wheel, and a second motor that is configured to roll the wheel.

Clause 2. The mobile medical platform of Clause 1, wherein: the first motor is configured to steer the wheel around a first axis that is substantially perpendicular to a plane corresponding to the first side of the rigid base, and the second motor is configured to roll the wheel around a second axis that is substantially parallel to the plane corresponding to the first side of the rigid base.

Clause 3. The mobile medical platform of Clause 2, wherein the first axis and the second axis are aligned to substantially eliminate a caster angle of the first axis.

Clause 4. The mobile medical platform of any of Clauses 1-3, wherein the respective wheel assembly further comprises a first spring that is positioned to exert a downward force on the wheel.

Clause 5. The mobile medical platform of any of Clauses 1-4, wherein the respective wheel assembly further comprises a second spring that is positioned to dampen relative movement between the wheel and the rigid base.

Clause 6. The mobile medical platform of any of Clauses 1-5, wherein the respective wheel assembly further comprises: a first spring; and a second spring located below the first spring, wherein the second spring is located above the wheel, and the first spring has a greater spring constant than a spring constant of the second spring.

Clause 7. The mobile medical platform of any of Clauses 1-6, further comprising:

one or more processors; and memory storing instructions, which, when executed by the one or more processors, cause at least one of the first motor or the second motor to move the wheel in accordance with one or more inputs.

Clause 8. The mobile medical platform of Clause 7, wherein the one or more wheel assemblies include at least two wheel assemblies, and the stored instructions, when executed by the one or more processors, cause the one or more processors to, in accordance with a determination that preset automatic-braking criteria are met, trigger respective first motors of one or more of the at least two wheel assemblies to steer the respective wheels of the at least two wheel assemblies into a preset braking configuration.

Clause 9. The mobile medical platform of Clause 8, wherein the one or more wheel assemblies include four wheel assemblies, and the preset braking configuration includes second axes of adjacent wheels of the four wheel assemblies being arranged at different angles.

Clause 10. The mobile medical platform of Clause 8 or 9, wherein the stored instructions, when executed by the one or more processors, cause the one or more processors to: trigger the at least two wheel assemblies to align respective wheels of the at least two wheel assemblies in a common direction in accordance with a determination that first criteria are met; and trigger the at least two wheel assemblies to place the respective wheels of the at least two wheel assemblies in a preset braking configuration in accordance with a determination that the first criteria are not met.

Clause 11. The mobile medical platform of Clause 10, wherein the first criteria include a requirement that an input of a first preset type is continuously maintained in order for the first criteria to be met.

Clause 12. The mobile medical platform of any of Clauses 7-11, wherein the stored instructions, when executed by the one or more processors, cause the one or more processors to: receive one or more control parameters corresponding to user input from one or more input devices that are in communication with the one or more processors; and control respective first motors and respective second motors of the one or more wheel assemblies to move respective wheels in accordance the one or more control parameters.

Clause 13. The mobile medical platform of Clause 12, wherein controlling the respective first motors and the respective second motors of the one or more wheel assemblies to move the respective wheels in accordance the one or more control parameters includes: controlling the first motor and the second motor of the respective wheel assembly to steer and roll the wheel of the respective wheel assembly at a same time.

Clause 14. The mobile medical platform of any of Clauses 7-13, wherein the stored instructions, when executed by the one or more processors, cause the one or more processors to: coordinate operations of two or more wheel assemblies to achieve a requested movement of the rigid base.

Clause 15. The mobile medical platform of Clause any of Clauses 1-14, further comprising: a robotic surgery system that is coupled to the rigid base, wherein the robotic surgery system includes a table top and one or more robotic arms that are configured to move relative to the table top.

Clause 16. A mobile medical platform, comprising: a rigid base; and one or more wheel assemblies that are coupled to a first side of the rigid base and support the rigid base, wherein a respective wheel assembly of the one or more wheel assemblies includes: a wheel that is configured to respectively rotate around a first axis and a second axis, the second axis being different from the first axis; and a first motor positioned for rotating the wheel around a respective one of the first axis and the second axis, wherein the first axis is aligned with the second axis that results in a negligible caster angle of the wheel.

Clause 17. The mobile medical platform of Clause 16, wherein: the first motor is positioned for rotating the wheel around the first axis; the respective wheel assembly of the one or more wheel assemblies further comprises a second motor positioned to rotate the wheel around the second axis; and the second axis is substantially parallel to a plane corresponding to a first side of the rigid base.

Clause 18. The mobile medical platform of Clause 16 or 17, wherein the respective wheel assembly of the one or more wheel assemblies further comprises a first spring positioned to exert a downward force on the wheel.

Clause 19. The mobile medical platform of any of Clauses 16-18, wherein the respective wheel assembly of the one or more wheel assemblies further comprises a second spring positioned to dampen relative movement between the wheel and the rigid base.

Clause 20. The mobile medical platform of any of Clauses 16-19, wherein the respective wheel assembly of the one or more wheel assemblies further comprises: a first spring; and a second spring located below the first spring, wherein the first spring has a greater spring constant than a spring constant of the second spring.

Clause 21. The mobile medical platform of any of Clauses 16-20, further comprising: one or more processors; and memory storing instructions, which, when executed by the one or more processors, cause the first motor to move the wheel in accordance with one or more inputs.

Clause 22. The mobile medical platform of Clause 21, wherein the one or more wheel assemblies include at least two wheel assemblies, and the stored instructions, when executed by the one or more processors, cause the one or more processors to, in accordance with a determination that preset automatic-braking criteria are met, trigger respective first motors of one or more of the at least two wheel assemblies to rotate respective wheels of the at least two wheel assemblies into a preset braking configuration.

Clause 23. The mobile medical platform of Clause 22, wherein the one or more wheel assemblies include four wheel assemblies, and the preset braking configuration includes second axes of adjacent wheels of the four wheel assemblies being arranged at different angles.

Clause 24. The mobile medical platform of any of Clauses 21-23, wherein the respective wheel assembly further includes a second motor, and the stored instructions, when executed by the one or more processors, cause the one or more processors to: receive one or more control parameters corresponding to user from one or more input devices that are in communication with the one or more processors; and control respective first motors and respective second motors of the one or more wheel assemblies to move respective wheels of the one or more wheel assemblies in accordance the one or more control parameters.

Clause 25. The mobile medical platform of any of Clauses 21-24, wherein: the one or more wheel assemblies includes at least two wheel assemblies; and the stored instructions, when executed by the one or more processors, cause the one or more processors to: trigger the at least two wheel assemblies to align respective wheels of the at least two wheel assemblies in a common direction in accordance with a determination that first criteria are met; and trigger the at least two wheel assemblies to place the respective wheels of the at least two wheel assemblies in a preset braking configuration in accordance with a determination that the first criteria are not met.

Clause 26. The mobile medical platform of Clause 25, wherein the first criteria include a requirement that an input of a first preset type is continuously maintained in order for the first criteria to be met.

Clause 27. The mobile medical platform of any of Clauses 21-26, wherein the respective wheel assembly further includes a second motor, and the stored instructions, when executed by the one or more processors, cause the one or more processors to: control the first motor and the second motor of the respective wheel assembly to rotate the wheel of the respective wheel assembly around the first axis and the second axis at the same time.

Clause 28. The mobile medical platform of any of Clauses 21-27, wherein: the one or more wheel assemblies includes at least two wheel assemblies; and the stored instructions, when executed by the one or more processors, cause the one or more processors to: coordinate operations of two or more wheel assemblies to achieve a requested movement of the rigid base.

Clause 29. The mobile medical platform of any of Clauses 16-28, further comprising: a robotic surgery system that is coupled to the rigid base, wherein the robotic surgery system includes a table top and one or more robotic arms that are configured to move relative to the table top.

Clause 30. A mobile medical platform, comprising: a rigid base; and one or more wheel assemblies that are coupled to a first side of the rigid base and support the rigid base during movement of the mobile medical platform, wherein a respective wheel assembly of the one or more wheel assemblies includes: a wheel; a first motor positioned for rotating the wheel; a first spring positioned to exert a downward force on the wheel; and a second spring positioned to dampen relative movement between the wheel and the rigid base.

Clause 31. The mobile medical platform of Clause 30, wherein: the second spring is located below the first spring; the second spring is located above the wheel; and the first spring has a greater spring constant than a spring constant of the second spring.

Clause 32. The mobile medical platform of Clause 30 or 31, wherein: the first motor is positioned for rotating the wheel around a first axis that is substantially perpendicular to a plane corresponding to the first side of the rigid base; and the respective wheel assembly of the one or more wheel assemblies further includes a second motor positioned for rolling the wheel around a second axis that is substantially parallel to the plane corresponding to the first side of the rigid base.

Clause 33. The mobile medical platform of Clause 32, wherein the first axis and the second axis are aligned to substantially eliminate a caster angle of the first axis.

Clause 34. The mobile medical platform of Clause 32 or 33, further comprising: one or more processors; and memory storing instructions, which, when executed by the one or more processors, cause at least one of the first motor or the second motor to move the wheel in accordance with one or more inputs.

Clause 35. The mobile medical platform of Clause 34, wherein the one or more wheel assemblies include at least two wheel assemblies, and the stored instructions, when executed by the one or more processors, cause the one or more processors to, in accordance with a determination that preset automatic-braking criteria are met trigger respective first motors of one or more of the at least two wheel assemblies to steer respective wheels of the at least two wheel assemblies into a preset braking configuration.

Clause 36. The mobile medical platform of Clause 35, wherein the one or more wheel assemblies include four wheel assemblies, and the preset braking configuration includes second axes of adjacent wheels of the four wheel assemblies being arranged at different angles.

Clause 37. The mobile medical platform of Clause 35 or 36, wherein: the one or more wheel assemblies includes at least two wheel assemblies; and the stored instructions, when executed by the one or more processors, cause the one or more processors to: trigger the at least two wheel assemblies to align the respective wheels of the at least two wheel assemblies in a common direction in accordance with a determination that first criteria are met; and trigger the at least two wheel assemblies to place the respective wheels of the at least two wheel assemblies in a preset braking configuration in accordance with a determination that the first criteria are not met.

Clause 38. The mobile medical platform of Clause 37, wherein the first criteria include a requirement that an input of a first preset type is continuously maintained in order for the first criteria to be met.

Clause 39. The mobile medical platform of any of Clauses 34-38, wherein the stored instructions, when executed by the one or more processors, cause the one or more processors to: receive one or more control parameters corresponding to user input from one or more input devices that are in communication with the one or more processors; and control the respective first motors and the respective second motors of the one or more wheel assemblies to move respective wheels in accordance the one or more control parameters.

Clause 40. The mobile medical platform of Clause 39, wherein controlling the respective first motors and the respective second motors of the one or more wheel assemblies to move the respective wheels in accordance the one or more control parameters includes controlling the first motor and the second motor of the respective wheel assembly to steer and roll the wheel of the respective wheel assembly at a same time.

Clause 41. The mobile medical platform of any of Clauses 34-40, wherein the stored instructions, when executed by the one or more processors, cause the one or more processors to coordinate operations of two or more wheel assemblies to achieve a requested movement of the rigid base.

Clause 42. The mobile medical platform of any of Clauses 30-41, further comprising: a robotic surgery system that is coupled to the rigid base, wherein the robotic surgery system includes a table top and one or more robotic arms that are configured to move relative to the table top.

Clause 43. A mobile medical platform, comprising: a rigid base; and at least four wheel assemblies that are coupled to the rigid base and support the rigid base, a respective wheel assembly of the at least four wheel assemblies including: a respective wheel; and a respective first motor positioned for steering the respective wheel; and one or more processors; and memory storing instructions, which, when executed by the one or more processors, cause steering of the respective wheels of the at least four wheel assemblies such that: the respective wheels of the at least four wheel assemblies are aligned in a common direction at a first time; and the respective wheels of the at least four wheel assemblies are arranged in a preset braking configuration, at a second time distinct from the first time, so that the rigid base is immobilized.

Clause 44. The mobile medical platform of Clause 43, wherein the respective wheels of the at least four wheel assemblies are directed to a common point while in the preset braking configuration.

Clause 45. The mobile medical platform of Clause 44, wherein the common point is a centroid of the at least four wheel assemblies.

Clause 46. The mobile medical platform of any of Clauses 43-45, wherein: the respective first motor is positioned for steering the respective wheel around a first axis that is substantially perpendicular to a plane corresponding to a first side of the rigid base; and the respective wheel assembly of the at least four wheel assemblies further includes a respective second motor positioned for rolling the respective wheel around a second axis that is substantially parallel to the plane corresponding to the first side of the rigid base.

Clause 47. The mobile medical platform of Clause 46, wherein the first axis and the second axis are aligned to substantially eliminate a caster angle of the first axis.

Clause 48. The mobile medical platform of Clause 46 or 47, wherein the stored instructions, when executed by the one or more processors, cause at least one of the respective first motor or the respective second motor to move the respective wheel in accordance with one or more inputs.

Clause 49. The mobile medical platform of any of Clauses 46-48, wherein the stored instructions, when executed by the one or more processors, cause the one or more processors to: receive one or more control parameters from one or more input devices that are in communication with the one or more processors; and control the respective first motors and the respective second motors of the one or more wheel assemblies to move the respective wheels in accordance the one or more control parameters.

Clause 50. The mobile medical platform of Clause 49, wherein controlling the respective first motors and the respective second motors of the one or more wheel assemblies to move the respective wheels in accordance the one or more control parameters includes controlling the respective first motor and the respective second motor of the respective wheel assembly to steer and roll the respective wheel of the respective wheel assembly at a same time.

Clause 51. The mobile medical platform of any of Clauses 43-50, wherein the respective wheel assembly of the at least four wheel assemblies further comprises a respective first spring that is positioned to exert a downward force on the respective wheel.

Clause 52. The mobile medical platform of any of Clauses 43-51, wherein the respective wheel assembly of the at least four wheel assemblies further comprises a respective second spring that is positioned to dampen relative movement between the respective wheel and the rigid base.

Clause 53. The mobile medical platform of any of Clauses 43-52, wherein the respective wheel assembly of the at least four wheel assemblies further comprises: a respective first spring; and a respective second spring located below the respective first spring, wherein the respective second spring is located above the respective wheel, and the respective first spring has a greater spring constant than a spring constant of the respective second spring.

Clause 54. The mobile medical platform of any of Clauses 43-53, wherein the stored instructions, when executed by the one or more processors, cause the one or more processors to, in accordance with a determination that preset automatic-braking criteria are met, trigger respective first motors of one or more of the at least four wheel assemblies to steer the respective wheels of the at least four wheel assemblies into the preset braking configuration.

Clause 55. The mobile medical platform of any of Clauses 43-54, wherein the preset braking configuration includes second axes of adjacent wheels of the four wheel assemblies being arranged at different angles.

Clause 56. The mobile medical platform of any of Clauses 43-55, wherein the stored instructions, when executed by the one or more processors, cause the one or more processors to coordinate operations of two or more wheel assemblies to achieve a requested movement of the rigid base.

Clause 57. The mobile medical platform of any of Clauses 43-56, wherein: the respective wheels of the at least four wheel assemblies are aligned in a common direction in accordance with a determination that first criteria are met; and the respective wheels of the at least four wheel assemblies are arranged in a preset braking configuration, in accordance with a determination that the first criteria are not met.

Clause 58. The mobile medical platform of Clause 57, wherein the first criteria include a requirement that an input of a first preset type is continuously maintained in order for the first criteria to be met.

Clause 59. The mobile medical platform of any of Clauses 43-58, further comprising a robotic surgery system that is coupled to the rigid base, wherein the robotic surgery system includes a table top and one or more robotic arms that are configured to move relative to the table top.

Clause 60. A method, comprising: at the mobile medical platform of any of Clauses 1-59: receiving user input to move the mobile medical platform; and moving at least one wheel of the one or more wheel assemblies, including any of: activating the first motor to orient the wheel in a respective direction corresponding to the user input; and activating the second motor to roll the wheel.

Clause 61. The method of Clause 60, wherein the second motor is activated after the wheel is oriented in the respective direction, and the wheel is rolled by the second motor while an orientation of the wheel is maintained in the respective direction.

Clause 62. The method of Clause 61, wherein the first motor and the second motor are activated at a same time.

Clause 63. The method of any of Clauses 60-62, wherein: activating the first motor to orient the wheel in a respective direction includes steering, by the first motor, the wheel around a first axis that is substantially perpendicular to a plane corresponding to the first side of the rigid base, and activating the second motor to roll the wheel includes powering the wheel, by the second motor, to roll around a second axis that is substantially parallel to the plane corresponding to the first side of the rigid base.

Clause 64. The method of Clause 63, wherein the first axis and the second axis are aligned to substantially eliminate a caster angle of the first axis.

Clause 65. The method of any of Clauses 60-64, wherein the wheel assembly further comprises a first spring that is positioned to exert a downward force on the wheel.

Clause 66. The method of any of Clauses 60-65, wherein the wheel assembly further comprises a second spring that is positioned to dampen relative movement between the wheel and the rigid base.

Clause 67. The method of any of Clauses 60-66, wherein: the wheel assembly further comprises a first spring and a second spring; the second spring is located above the wheel and below the first spring; and the first spring has a greater spring constant than a spring constant of the second spring.

Clause 68. The method of any of Clauses 60-67, wherein the one or more wheel assemblies includes at least two wheel assemblies, the method further comprising: in accordance with a determination that first criteria are met, triggering at least two wheel assemblies of the one or more wheel assemblies to align the respective wheels of the at least two wheel assemblies in a common direction; and in accordance with a determination that the first criteria are not met, triggering respective first motors of one or more of the at least two wheel assemblies to steer the respective wheels of the at least two wheel assemblies into a preset braking configuration.

Clause 69. The method of Clause 68, wherein: the one or more wheel assemblies include four wheel assemblies; and triggering respective first motors of one or more of the at least two wheel assemblies to steer the respective wheels of the at least two wheel assemblies into a preset braking configuration includes rotating, by the respective first motors, the respective wheels around axes of adjacent wheels of the four wheel assemblies such that axes of the adjacent wheels of the four wheel assemblies are arranged at different angles.

Clause 70. The method of Clause 68 or 69, wherein the first criteria include a requirement that an input of a first preset type is continuously maintained in order for the first criteria to be met.

Clause 71. The method of any of any of Clauses 60-70, wherein the user input to move the mobile medical platform is received from one or more input devices.

Clause 72. The method of any of Clauses 60-71, further comprising: coordinating operations of two or more wheel assemblies to achieve a requested movement of the rigid base, the requested movement of the rigid base corresponding to the user input.

Clause 73. The method of any of Clauses 60-72, wherein the mobile medical platform further includes a robotic surgery system that is coupled to the rigid base and the robotic surgery system includes a table top and one or more robotic arms, the method further comprising moving the one or more robotic arms relative to the table top.

Clause 74. A method, comprising: utilizing the mobile medical platform of any of Clauses 1-59, wherein the mobile medical platform includes at least two wheel assemblies for: receiving input to move the mobile medical platform from one or more input devices; and generating one or more control instructions for controlling respective first motors and respective second motors of the at least two wheel assemblies, including: triggering the at least two wheel assemblies to align respective wheels of the at least two wheel assemblies in a common direction in accordance with a determination that the input meets first criteria; and triggering the at least two wheel assemblies to place the respective wheels of the at least two wheel assemblies in a preset braking configuration in accordance with a determination that the input meets second criteria different from the first criteria.

Clause 75. The method of Clause 74, wherein triggering the at least two wheel assemblies to align the respective wheels of the at least two wheel assemblies in a common direction in accordance with a determination that the input meets first criteria further includes: activating the respective first motors to align the respective wheels of the at least two wheel assemblies in a common direction; and activating a respective second motor to roll the respective wheels, wherein the first motor and the second motor are activated at a same time.

Clause 76. The method of Clause 74 or 75, wherein triggering the at least two wheel assemblies to align the respective wheels of the at least two wheel assemblies in a common direction in accordance with a determination that the input meets first criteria further includes: activating the respective first motors to align the respective wheels of the at least two wheel assemblies in a common direction; and activating the respective second motors to roll the respective wheels after the respective wheels are maintained in the respective direction and while an orientation of the respective wheels are maintained in the respective direction.

Clause 77. The method of Clause 76, wherein: activating the respective first motors to align the respective wheels of the at least two wheel assemblies in a common direction includes steering, by the respective first motors, the respective wheels around respective first axes that are substantially perpendicular to a plane corresponding to the first side of the rigid base, and activating the respective second motors to roll the respective wheels includes powering the wheel, by the respective second motors, to roll around respective second axes that are substantially parallel to the plane corresponding to the first side of the rigid base.

Clause 78. The method of Clause 77, wherein a respective first axis and respective second axis of a respective wheel of the at least two wheel assemblies are aligned to substantially eliminate a caster angle of the respective first axis.

Clause 79. The method of Clause 77 or 78, wherein: the one or more wheel assemblies include at least four wheel assemblies; and triggering the at least four wheel assemblies to place the respective wheels of the at least four wheel assemblies in the preset braking configuration includes rotating, by the respective first motors, the respective wheels around first axes of adjacent wheels of the four wheel assemblies such that the second axes of the adjacent wheels of the four wheel assemblies are arranged at different angles.

Clause 80. The method of any of Clauses 74-79, wherein a respective wheel assembly of the at least two wheel assemblies further comprises a respective first spring that is positioned to exert a downward force on the respective wheel.

Clause 81. The method of any of Clauses 74-80, wherein a respective wheel assembly of the at least two wheel assemblies further comprises a respective second spring that is positioned to dampen relative movement between the respective wheel and the rigid base.

Clause 82. The method of any of Clauses 74-81, wherein: the a respective wheel assembly of the at least two wheel assemblies further comprises a respective first spring and a respective second spring; the respective second spring is located above the respective wheel and below the respective first spring; and the respective first spring has a greater spring constant than a spring constant of the respective second spring.

Clause 83. The method of any of Clauses 74-82, wherein the first criteria include a requirement that an input of a first preset type is continuously maintained in order for the first criteria to be met.

Clause 84. The method of any of Clauses 74-83, further comprising: coordinating operations of the at least two wheel assemblies to achieve a requested movement of the rigid base, the requested movement of the rigid base corresponding to the input.

Clause 85. The method of any of Clauses 74-84, wherein the mobile medical platform further includes a robotic surgery system that is coupled to the rigid base and the robotic surgery system includes a table top and one or more robotic arms, the method further comprising moving the one or more robotic arms relative to the table top.

1. A mobile medical platform, comprising:
a rigid base; and
one or more wheel assemblies that are coupled to a first side of the rigid base to support and move the rigid base in a physical environment, wherein a respective wheel assembly of the one or more wheel assemblies includes:
a wheel,
a first motor that is configured to steer the wheel, and
a second motor that is configured to roll the wheel.

2. The mobile medical platform of claim 1, wherein:
the first motor is configured to steer the wheel around a first axis that is substantially perpendicular to a plane corresponding to the first side of the rigid base, and
the second motor is configured to roll the wheel around a second axis that is substantially parallel to the plane corresponding to the first side of the rigid base.

3. The mobile medical platform of claim 2, wherein the first axis and the second axis are aligned to substantially eliminate a caster angle of the first axis.

4. The mobile medical platform of claim 1, wherein the respective wheel assembly further comprises a first spring that is positioned to exert a downward force on the wheel.

5. The mobile medical platform of claim 1, wherein the respective wheel assembly further comprises a second spring that is positioned to dampen relative movement between the wheel and the rigid base.

6. The mobile medical platform of claim 1, wherein the respective wheel assembly further comprises:
a first spring; and
a second spring located below the first spring, wherein the second spring is located above the wheel, and the first spring has a greater spring constant than a spring constant of the second spring.

7. The mobile medical platform of claim 1, further comprising:
one or more processors; and
memory storing instructions, which, when executed by the one or more processors, cause at least one of the first motor or the second motor to move the wheel in accordance with one or more inputs.

8. The mobile medical platform of claim 7, wherein the one or more wheel assemblies include at least two wheel assemblies, and the stored instructions, when executed by the one or more processors, cause the one or more processors to, in accordance with a determination that preset automatic-braking criteria are met, trigger respective first motors of one or more of the at least two wheel assemblies to steer the respective wheels of the at least two wheel assemblies into a preset braking configuration.

9. The mobile medical platform of claim 8, wherein the one or more wheel assemblies include four wheel assemblies, and the preset braking configuration includes second axes of adjacent wheels of the four wheel assemblies being arranged at different angles.

10. The mobile medical platform of claim 8, wherein the stored instructions, when executed by the one or more processors, cause the one or more processors to:
trigger the at least two wheel assemblies to align respective wheels of the at least two wheel assemblies in a common direction in accordance with a determination that first criteria are met; and
trigger the at least two wheel assemblies to place the respective wheels of the at least two wheel assemblies in a preset braking configuration in accordance with a determination that the first criteria are not met.

11. The mobile medical platform of claim 10, wherein the first criteria include a requirement that an input of a first preset type is continuously maintained in order for the first criteria to be met.

12. The mobile medical platform of claim 7, wherein the stored instructions, when executed by the one or more processors, cause the one or more processors to:
receive one or more control parameters corresponding to user input from one or more input devices that are in communication with the one or more processors; and
control respective first motors and respective second motors of the one or more wheel assemblies to move respective wheels in accordance the one or more control parameters.

13. The mobile medical platform of claim 12, wherein controlling the respective first motors and the respective second motors of the one or more wheel assemblies to move the respective wheels in accordance the one or more control parameters includes:
controlling the first motor and the second motor of the respective wheel assembly to steer and roll the wheel of the respective wheel assembly at a same time.

14. The mobile medical platform of claim 7, wherein the stored instructions, when executed by the one or more processors, cause the one or more processors to:
coordinate operations of two or more wheel assemblies to achieve a requested movement of the rigid base.

15. The mobile medical platform of claim 1, further comprising:
a robotic surgery system that is coupled to the rigid base, wherein the robotic surgery system includes a table top and one or more robotic arms that are configured to move relative to the table top.

16-59. (canceled)

60. A method, comprising:
at a mobile medical platform including a rigid base and one or more wheel assemblies that are coupled to a first side of the rigid base to support and move the rigid base in a physical environment, wherein a respective wheel assembly of the one or more wheel assemblies includes a wheel, a first motor that is configured to steer the wheel, and a second motor that is configured to roll the wheel:
receiving user input to move the mobile medical platform; and
moving at least one wheel of the one or more wheel assemblies, including any of:
activating the first motor to orient the wheel in a respective direction corresponding to the user input; and
activating the second motor to roll the wheel.

61. The method of claim 60, wherein:
the second motor is activated after the wheel is oriented in the respective direction, and the wheel is rolled by the second motor while an orientation of the wheel is maintained in the respective direction; and
the first motor and the second motor are activated at a same time.

62. (canceled)

63. The method of claim 60, wherein:
activating the first motor to orient the wheel in a respective direction includes steering, by the first motor, the wheel around a first axis that is substantially perpendicular to a plane corresponding to the first side of the rigid base;
activating the second motor to roll the wheel includes powering the wheel, by the second motor, to roll around a second axis that is substantially parallel to the plane corresponding to the first side of the rigid base; and
the first axis and the second axis are aligned to substantially eliminate a caster angle of the first axis.

64. (canceled)

65. (canceled)

66. (canceled)

67. (canceled)

68. The method of claim 60, wherein the one or more wheel assemblies includes at least two wheel assemblies, the method further comprising:
in accordance with a determination that first criteria are met, triggering at least two wheel assemblies of the one or more wheel assemblies to align the respective wheels of the at least two wheel assemblies in a common direction; and
in accordance with a determination that the first criteria are not met, triggering respective first motors of one or more of the at least two wheel assemblies to steer the respective wheels of the at least two wheel assemblies into a preset braking configuration.

69. The method of claim 68, wherein:
the one or more wheel assemblies include four wheel assemblies; and
triggering respective first motors of one or more of the at least two wheel assemblies to steer the respective wheels of the at least two wheel assemblies into a preset braking configuration includes rotating, by the respective first motors, the respective wheels around axes of adjacent wheels of the four wheel assemblies such that axes of the adjacent wheels of the four wheel assemblies are arranged at different angles.

70-85. (canceled)
